(19) United States
(12) Patent Application Publication
(10) Pub. No.: US 2022/0308061 A1
(43) Pub. Date: Sep. 29, 2022

(54) METHOD TO SEQUENCE MRNA IN SINGLE CELLS IN PARALLEL WITH QUANTIFICATION OF INTRACELLULAR PHENOTYPE

(71) Applicant: The Regents of the University of California, Twelfth Floor, CA (US)

(72) Inventors: Owen N. Witte, Sherman Oaks, CA (US); Pavlo A. Nesterenko, Los Angeles, CA (US); Jami McLaughlin Witte, Sherman Oaks, CA (US)

(73) Assignee: The Regents of the University of California, Oakland, CA (US)

(21) Appl. No.: 17/638,092

(22) PCT Filed: Sep. 2, 2020
(86) PCT No.: PCT/US20/49055

§ 371 Date: Feb. 24, 2022

Related U.S. Application Data
(60) Provisional application No. 63/035,161, filed on Jun. 5, 2020.
(60) Provisional application No. 62/895,382, filed on Sep. 3, 2019.

(51) Int. Cl. G01N 33/574; C12Q 1/6804; G01N 33/569; C12Q 1/6806

(52) U.S. Cl. CPC G01N 33/57484; C12Q 1/6804; G01N 33/56972; C12Q 1/6806; G01N 2015/149

(57) ABSTRACT

The disclosure provides methods and materials useful for obtaining novel TCR gene sequences that are useful in tumor-specific T cell receptor (TCR) gene transfer. Embodiments of the invention also include methods of crosslinking and permeabilizing mammalian cells that can, for example, be used in methods for obtaining novel TCR gene sequences. The disclosure further provides methods and materials useful for obtaining novel TCR gene sequences. Tumor-specific T cell receptor gene transfer enables specific and potent immune targeting of tumor and viral antigens, and for this reason is technology of significant interest to medical personnel.

### CROSS-REFERENCE TO RELATED APPLICATIONS

This application claims the benefit under 35 U.S.C. Section 119(e) of U.S. Provisional Patent Application Ser. No. 62/895,382, filed on Sep. 3, 2019, and U.S. Provisional Patent Application Ser. No. 63/035,161, filed on Jun. 5, 2020, which applications are incorporated in their entirety by reference herein.

### STATEMENT REGARDING FEDERALLY SPONSORED RESEARCH AND DEVELOPMENT

This invention was made with government support under Grant Number CA233074, awarded by the National Institutes of Health. The government has certain rights in the invention.

### TECHNICAL FIELD

The present invention relates to methods and materials useful for sequencing polynucleotides such as those encoding αβ T cell receptors.

### BACKGROUND OF THE INVENTION

The α/β T cell receptor (TCR) determines the unique specificity of each naïve T cell. Upon assembly with CD3 signaling proteins on the T cell surface, the TCR surveils peptide ligands presented by major histocompatibility complex (MHC) molecules on the surface of nucleated cells. The specificity of the TCR for a peptide-MHC complex is determined by both the presenting MHC molecule and the presented peptide. The MHC locus (also known as the human leukocyte antigen (HLA) locus in humans) is the most multi-allelic locus in the human genome, comprising >18,000 MHC class I and II alleles that vary widely in frequency across ethnic subgroups. Ligands presented by MHC class I molecules are derived primarily from proteasomal cleavage of endogenously expressed antigens. Infected and cancerous cells present peptides that are recognized by CD8⁺ T cells as foreign or aberrant, resulting in T cell-mediated killing of the presenting cell.

T cells can be engineered to kill tumor cells through the transfer of tumor-reactive αβ TCR genes. However, implementation of personalized TCR gene therapy is complicated by the need to identify new reactive TCRs, and to genetically modify patient T cells on-demand. This is challenging for tumors that cannot be accessed for sequencing and for low mutational burden tumors such as those with few or no neoantigens. Particularly for these last tumor types, targeting public (non-patient specific), tumor-restricted antigens with off-the-shelf TCRs remains an attractive option.

For the reasons noted above, there is a need in the art for additional methods and materials useful for selecting and sequencing polynucleotides such as those encoding αβ T cell receptors.

### SUMMARY OF THE INVENTION

Antigen specific T cells can be quantified and characterized by their cytokine production profiles. Building upon this knowledge, we have discovered that intracellular cytokine staining allows for identification of antigen specific T cells with enhanced specificity and reliability as compared to surface activation markers. This discovery has allowed the design of new methods that are useful, for example, for selectively obtaining new T cell receptor encoding polynucleotides from single CD8⁺ Cytotoxic T cells. This is important because cytotoxic T cells are effective at clearing infections as well as cancerous cells when appropriately targeted through the T cell receptor. Consequently, using embodiments of the invention disclosed herein, T lymphocytes can be engineered to express pathogen or tumor-specific T cell receptor genes and thereby kill infected or cancerous cells.

As discussed below, we have developed methods for crosslinking cells in a manner that facilitates functional profiling and polynucleotide cloning, as well as methods for obtaining antigen specific T cell receptor genes that are useful in targeted immunotherapies.

Embodiments of the invention include methods of crosslinking mammalian cells. Typically these methods comprise combining the mammalian cells with a permeabilization agent and a chemically cleavable crosslinker selected to have an ability to couple intracellular polypeptides to mRNA under a first set of conditions and further release the cellular polypeptides from mRNA under a second set of conditions (e.g. via the addition of a reducing agent); so that the mammalian cells are crosslinked. In certain embodiments of the invention, the methods comprise combining fixed and permeabilized mammalian cells with intracellular staining reagents such as fluorescent antibodies. Such fluorescent antibodies can be directed to one or more target polypeptides within the fixed and permeabilized mammalian cells so that fluorescent activated cell sorting can be performed to select one or more fixed and permeabilized mammalian cells containing the one or more target polypeptides; followed by sequencing one or more mRNAs present in the one or more selected mammalian cells.

Typically these crosslinking methods comprise one or more additional steps that can include, for example, releasing the cellular polypeptides from mRNA (e.g. via the addition of a reducing agent); and/or encapsulating the mammalian cells within fluid droplets (e.g. in an oil and water emulsion); and/or combining the mammalian cells with a bead comprising a barcode; and/or obtaining the sequences of one or more mRNAs present in one or more selected dead mammalian cells using a dynamic microfluidic system. Embodiments of the invention include compositions of matter comprising polynucleotides that are selected by such methods.

Embodiments of the invention also include methods for obtaining polynucleotides encoding Vα and Vβ T cell receptor polypeptides. These methods typically comprise combining together antigen. T cells and a cytokine secretion inhibitor under conditions selected to activate the T cells in response to the antigen, and then fixing and permeabilizing activated T cells. These fixed and permeabilized T cells are then combined with fluorescent antibodies directed to one or more polypeptides such as cytokines, or other molecules such as nuclear transcription factors present within T cells of interest. Fluorescent activated cell sorting is then performed to select one or more cells containing the one or more cytokines or other molecules observed to be produced in, for example, activated T cells. In these methods, polynucleotides encoding Vα and Vβ T cell receptor polypeptides are then obtained from the selected one or more cells. Embodiments of the invention also include compositions of matter comprising polynucleotides encoding Vα and Vβ T cell receptor polypeptides produced by a method disclosed herein.

In illustrative working embodiments of the invention disclosed herein, T cells are combined with antigen in the presence of the secretion inhibitor Brefeldin A, so that the cytokines TNFα and IFNγ are not secreted from the T cells. Subsequently, these T cells are fixed, permeabilized and then stained with fluorescent antibodies to TNFα and IFNγ, followed by fluorescent activated cell sorting (FACS). These methods are designed to perform intracellular staining while simultaneously preserving human TCR mRNA quality at the single cell level. This technique allows for single-cell FACS deposition of human cytokine producing cells, followed with TCR mRNA paired TCR alpha and beta chain sequencing. Using such methods, we are able to generate cDNA sequences covering the variable regions of the TCR, which then allows reconstruction of the full length heterologous TCRs for use in gene therapy.

The power and broad applicability of this approach has been confirmed with multiple peptide antigens, including well described viral epitopes from cytomegalovirus and Epstein-Barr virus. We have shown that we can capture human, intracellularly stained T cells and single-cell sequence TCR alpha and beta pair cDNA. Identified clones have been cloned into retrovirus vectors and tested for reactivity to the cognate peptide in donor PBMCs. Further, TCR clones have been tested for ability to kill target cell lines expressing full length CMV protein. Significantly, TCR clones obtained from illustrative embodiments of the invention are able to successfully recognize endogenous processed peptide and kill target cell lines at efficiencies comparable to clinical grade TCRs used in adoptive cell therapies.

Embodiments of the invention disclosed herein can be expanded to other aspects of T cell biology where small subsets of T cell are identified by an intracellular marker. For example, nuclear transcription factors identify specific subtypes of T cells and it is of interest to identify their TCRs. Embodiments of the invention can be used to clone TCRs of low frequency T cells identified by a transcription factor phenotype. Further, FACS is now capable to discern up to 18 fluorophores, thus embodiments of the invention allow for highly multiplexed analysis of fine T cell subsets. These subsets can be identified by combinations of, for example, nuclear transcription factors, cytokines and the like. Embodiments of the methods disclosed herein provide a new avenue for the discovery of TCRs to defined antigens as well as discovery of novel reactivities defined by phenotype.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 shows TCR construct overexpression allows for TCR mRNA sequencing post intracellular staining. Donor PBMCs are transduced with TCR F5 that, which recognizes MART1 HLA-A2 restricted epitope (left panel). Clone F5 overexpressing populations are then either stained with cognate tetramer or stimulated with cognate peptide (ELAGIGILTV (SEQ ID NO: 1)) and subsequently stained for intracellular TNFα and IFNγ (middle panel). Responding cells are then single-cell FACS deposited for cloning. TCR cDNA is prepared via RT-PCR and subsequently analyzed by Sanger sequencing (right panel).

FIGS. 2A-2D show TCR alpha and beta pairs can be recovered in primary T cells post intracellular staining. FIG. 2A shows as schematic for TCR mRNA sequencing post-intracellular staining of primary T cells. PBMCs are cultured for 9 days in the presence of antigenic peptide: (NLVPMVATV (SEQ ID NO: 2), CMV) (GLCTLVAML (SEQ ID NO: 3), EBV) and 25 U/ml IL2. Then cells are washed and rested in media for 12 hours, followed by peptide re-stimulation in the presence of Brefeldin A to inhibit cytokine secretion. T cells are then stained for intracellular TNFα and IFNγ and FACS sorted for single-cell cloning. FIG. 2B shows CMV+ and EBV+ subject PBMC processed as show in panel a, stimulated with CMV or EBV peptide and stained for Intracellular cytokines TNFα and IFNγ. These PBMCs are also stained with cognate HLA-A2 tetramers and activated with peptide and stained with CD137 post activation. Cells that are responsive (green box) are single-cell cloned for TCR alpha and beta. FIG. 2C shows a summary table of TCR clones recovered by each technique and frequency of recovery within each technique. FIG. 2D shows Cloning efficiency of cells in panel b, defined by frequency of successful recovery paired TCR alpha and beta chains.

FIGS. 3A-3C show Intracellular staining for TNFα and IFNγ identified antigen specific TCRs from primary T cells. FIG. 3A shows a Schematic for TCR clone functional testing in normal donor PBMC. TCR clone (alpha and beta pair) retrovirus constructs are transduced into PBMCs activated with CD3/28 dynabeads for 48 hours. Transduction is evaluated by murine V beta expression and cognate tetramer staining, HLA-A2-pp65. FIG. 3B shows Cell preparations from panel are stimulated with PC3 cell line which is either engineered to overexpress HLA-A2 and CMV pp65. Supernatant is collected 48 hours later and amount of IFNγ is quantified by ELISA. FIG. 3C shows T cell preparations from panel are tested for their ability to kill target cells by cocultured with PC3 cell line that over express HLA-A2 and CMV pp65. Cell viability is tracked by monitoring GFP level in PC3 cell line.

FIGS. 4A-4B show the DSP chemical structure (FIG. 4A); and Human PBMCs stimulated with PMA/Ionomycin and stained for intracellular IFNγ and TNFα under different conditions of permeabilization with DSP and triton X-100 (FIG. 4B).

FIGS. 5A-5E shows Human PBMCs and Jurkat cells are mixed at 5:1 ratio and permeabilized with DSP and Triton X-100. These treated cells are then submitted for 10× genomics TCR V(D)J sequencing. Separate samples of live and paraformaldehyde (PFA) permeabilized cells are processed in the same run as a positive and negative controls. FIGS. 5A and 5B show Electrophoresis cDNA analysis after PCR amplification. FIG. 5C shows TCR sequence analysis after next generation sequencing FIG. 5D shows TCR clonotypes as shown in the Loupe VDJ Browser (10× Genomics). FIG. 5E shows an analysis of clonotype overlap between live and DSP permeabilized samples.

FIG. 6 provides a schematic of a pipeline to engineer personalized adoptive cell therapy using CLint-Seq.

FIGS. 7A-7B. show Intracellular staining identifies antigen specific T cells with a lower rate of false positives than CD137 activation marker. FIG. 7A shows a Schematic for experimental design to compare antigen specific activation to bystander T cell activation. Donor PBMC are transduced with a NY-ESO TCR (clone 1G4) construct. Resultant populations are spiked into untransduced population of cells that was treated similarly. Dilution is confirmed by secondary transduction marker staining as well as tetramer staining. FIG. 7B shows T cell populations from previous panel are stimulated with peptide and then stained for CD137. Alternatively, cells are stained intracellularly for IFNγ and TNFα. Truly reactive cells are NGFR⁺ and CD137⁺ or IFNγ⁺/TNFα⁺. Repeat of this experiment yielded a similar result.

FIGS. 8A-8D show how CLint-Seq allows for single-cell mRNA sequencing in droplet-based format. FIG. 8A shows a Schematic for tethering of cellular mRNA to cellular protein mass via DSP. The reducing reagents present in the drop-seq fluidics allow the untethering of mRNA and subsequent RT-PCR. FIG. 8B shows Activated human PBMCs and Jurkat cells are mixed at 5:1 ratio, subsequently fixed with DSP and permeabilized with Triton X-100. These treated cells are then submitted for 10× genomics TCR V(D)J sequencing. Separate samples of live and PFA permeabilized cells are processed in the same run as a positive and negative controls. Subsequently, cDNA libraries are analyzed be electrophoresis.

FIG. 8C shows TCR clone metadata analysis after next generation sequencing FIG. 8D shows Pie chart analysis of TCR diversity of all clones reported in the Loupe VDJ browser (10× Genomics).

FIGS. 9A-9D show CLint-Seq coupled to droplet-based sequencing recovers EBV specific TCRs. FIG. 9A shows how Human PBMCs are co-cultured with EBV 9mer epitopes, then re-stimulated in the presence of EBV peptide and Brefeldin A and subsequently stained for TNFα and IFNγ cytokines. DSP is used as a crosslinker. Responding cells are FACS sorted into a 2 ml Eppendorf tube and submitted for 10× Genomics V(D)J analysis. FIG. 9B shows Metadata for the 10× Genomics TCR sequencing done using CLint-Seq as well as a historical control generated with tetramer selection. FIG. 9C shows EBV clonotypes generated by CLint-Seq were filtered for clones with alpha/beta pair and frequency of 2 or more. The resultant set was compared to the tetramer clones filtered in the same way to determine overlap between techniques. FIG. 9D shows Frequency distribution of clonotypes that were found by both techniques.

FIGS. 10A-IOC show analysis of TNFα and IFNγ identified antigen specific TCRs from primary human T cells. FIG. 10A shows a Schematic for TCR functional testing in healthy donor PBMCs. CMV-reactive TCRs identified by ICS were cloned into retroviral constructs and used to transduce PBMCs activated with Dynabeads for 48 hours. TCR specificity was evaluated by cognate tetramer staining for CMV pp65 (NLVPMVATV). FIG. 10B shows how TCR-transduced PBMCs were stimulated with PC3 cells engineered to express HLA-A2 with or without CMV pp65. Cell supernatants were collected 48 hours after co-culture and secreted IFNγ quantified by ELISA. Error bars represent standard deviation. FIG. 10C shows the Cytotoxicity of ICS-identified CMV TCRs was evaluated by coculturing TCR-transduced T cells with GFP⁺ PC3 cells expressing HLA-A2 and CMV pp65. Relative viability was measured by GFP fluorescence using the Incucyte system. Data are representative of two independent experiments.

FIG. 11A-11B show how intracellular profile selection allows for TCR recovery from human regulatory T cells by intra-nuclear profiling of FOXP3 protein. FIG. 11A shows ICS analysis in Treg cells. CD4⁺ PBMCs are expanded in-vitro for 9 days and then stained for surface antigens (CD3, CD4, CD8, CD25), fixed and permeabilized, and stained for FOXP3. Single Treg cells (CD3⁺, CD4⁺, CD8⁻, CD25⁺, FOXP3⁺) were FACS deposited into % well plates and RT-PCR is performed for TCR sequencing. Cloning efficiency is reported as frequency of successful recovery of full length TCR alpha and beta pairs. FIG. 11B shows ICS analysis is performed on 40 cells and 33 alpha/beta TCR pairs are generated. Five of the TCRs sequenced are shown are shown.

FIGS. 12A-12D show Gating hierarchies. FIG. 12A shows a Gating hierarchy used for FACS sorting PBMCs based on CD137 and tetramer staining.

FIG. 12B shows Gating hierarchy used for FACS sorting of PBMCs based on Intracellular staining. FIG. 12C shows a Gating hierarchy used to analyze CD137 staining. FIG. 12D shows a Gating hierarchy for FoxP3 level analysis and FACS sorting of T regulatory cells.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

T cells redirected by genetic introduction of a cancer specific T cell receptor (TCR) can mediate regression of late stage tumors (1). To translate these advances to other cancers the field needs to identify target proteins and corresponding TCRs. TCR alpha and beta heterodimer binds antigenic peptide presented on surface of MHC, which leads to T cell activation. TCRs for use in adoptive cell therapies are usually cloned from mRNA in human T cells specific for antigen of interest.

Antigen specific T cells can be identified by direct staining of the TCR by soluble peptide-MHC constructs, commonly known as pMHC tetramers (2). Tetramer reactive cells can then be sorted for TCR cloning. Generation of tetramer reagents is time consuming and requires the knowledge of the peptide (2). Alternatively, T cells can be activated with complex mixtures of peptides and activated cells can be isolated based on expression of activation markers by Fluorescence Activated Cell Sorting (FACS) (3, 4). To preserve the quality of the TCR mRNA this analysis has been limited to those activation markers found on the cell surface. Such that live cells can be used for downstream TCR cloning. CD137 is a marker that is upregulated post TCR signaling on cell surface and has been used to clone reactive T cells (3). CD107 is a degranulation marker, which is upregulated when the activated T cell transports vesicles to the cell surface (4).

Most common immunological tool to quantify T cells and assess effector functions is intracellular cytokine staining (ICS) for effector cytokines such as TNFα and IFNγ (5-7). During ICS T cells are stimulated with antigen in the presence of secretion inhibitors, such that effector cytokines are produced but remain inside the cell (5, 8). Subsequently cells are fixed and permeabilized to allow for intracellular staining with antibodies. Cytokine producing cells are then analyzed via FACS. ICS has not been used for TCR cloning as fixation is thought to degrade mRNA. An alternative technique allows for cytokine analysis in live cells (H. Brosterhus, et al., Enrichment and detection of live antigen—specific CD4+ and CD8+ T cells based on cytokine secretion. Eur. J. Immunol. 29, 4053-4059 (1999)). This is a complex technique where a lymphocyte specific reagent captures cytokines as they are being secreted at the cell surface.

Here we describe techniques which allow for TCR cloning from cells that have been fixed and permeabilized such that ICS can be performed. We adapt a published method for deep sequencing analysis of mRNA in neuronal cells that have been fixed and permeabilized (9, 10). The method we developed robustly works for multiple antigens and allows for cloning of antigen specific TCRs.

Embodiments of the invention include methods of crosslinking a wide variety of different mammalian cells. Typically, these methods comprise combining the mammalian cells with a chemically cleavable crosslinker selected to couple intracellular polypeptides to mRNA (e.g. so that mRNA is coupled to the polypeptides via amine groups). In this context, there is a large family of cleavable crosslinking reagents that can be used in methods of the invention (e.g. those comprising S—S linkages that can be cleaved by a reducing agent). Such crosslinkers can vary in chain length and primary reactivities and include, for example, Lomant's Reagent, DTBP Dimethyl 3,3′-dithiobispropionimidate, DST disuccinimidyl tartrate, EGS ethylene glycolbis (succinimidylsuccinate), SCNE di-6-(3-succinimidyl carbonyloxymethyl-4-nitro-phenoxy)-hexanoic acid disulfide diethanol ester and the like. Illustrative but non limiting examples of cleavable crosslinkers are described in Xiang et al., Nucleic Acids Research, 2004. Vol. 32, No. 22; Mattson et al., Molecular Biology Reports 17: 167-183, 1993; and Wang et al., Bioconjugate Chem. 2012, 23, 705-713, the contents of which are incorporated herein by reference. Such crosslinkers are available from a number of commercial sources, such as ThermoFisher Scientific (see, e.g. the ThermoFisher Scientific catalog which is incorporated herein by reference).

In illustrative working embodiments of the invention, the chemically cleavable crosslinker is Lomant's Reagent, 3,3′-Dithiodipropionic acid di(Nhydroxysuccinimide ester, which is a water-insoluble, homo-bifunctional N-hydroxysuccimide ester (NHS ester) crosslinker that is thiol-cleavable, primary amine-reactive. DSP contains an amine-reactive NHS ester at each end of an 8-carbon spacer arm. NHS esters react with primary amines at pH 7-9 to form stable amide bonds and releasing N-hydroxy-succinimide. Proteins generally have several primary amines in the side chain of lysine (K) residues and the N-terminus of each polypeptide that are available as targets for NHS ester crosslinking reagents.

In certain embodiments of the invention, the methods comprise combining the fixed and permeabilized mammalian cells with fluorescent antibodies directed to one or more target polypeptides (e.g. FOXP3) within the fixed and permeabilized mammalian cells; performing fluorescent activated cell sorting to select one or more fixed and permeabilized mammalian cells containing the one or more target polypeptides; and then sequencing one or more mRNAs present in the one or more selected mammalian cells.

Typically these crosslinking methods comprise one or more steps that include releasing the cellular polypeptides from mRNA (e.g. under reducing conditions); and/or encapsulation of the mammalian cells within fluid droplets; and/or combining the mammalian cells with a bead comprising a barcode; and/or obtaining the sequences of one or more mRNAs present in one or more selected dead mammalian cells using a dynamic microfluidic system (e.g. droplet based scRNA-seq systems such as those disclosed in Salomon et al., Lab Chip, 2019, 19, 1706, which is incorporated herein by reference). In illustrative embodiments of the invention, T cells are combined with a chemically cleavable crosslinker selected to couple an intracellular polypeptide to mRNA (e.g. so that mRNA is coupled to a protein with in FOXP3 expressing cells etc.); and then release the nuclear factor from mRNA under reducing conditions. In certain embodiments of the invention, the chemically cleavable crosslinker comprises 3,3′-Dithiodipropionic acid di(Nhydroxysuccinimide ester). Embodiments of the invention include compositions of matter comprising polynucleotides generated in such methods of crosslinking and permeabilizing mammalian cells.

Embodiments of the invention also include methods for obtaining polynucleotides encoding Vα and Vβ T cell receptor polypeptides. These methods typically comprise combining together antigen (e.g. PAP or another cancer antigen that is presented by an antigen presenting cell), T cells and a cytokine secretion inhibitor under conditions selected to activate the T cells in response to the antigen, and then fixing and permeabilizing activated T cells. In the methods disclosed herein, the fixed and permeabilized T cells are further combined with fluorescent antibodies directed to one or more cytokines or other molecules observed to be produced T cells of interest. In these methods, fluorescent activated cell sorting can then be performed to select one or more cells containing the one or more cytokines or other molecules observed to be produced in T cells of interest. In this context, the methods disclosed herein can be generally adapted for use with a wide range of methods and materials in this art, for example those disclosed in, U.S. Patent Publication Nos. 201502752%, 20150203886, 201502752%, 20180223275, 20180073013, and 20200182884, the contents of which are incorporated by reference.

Because FACS is capable to discern up to 18 fluorophores, embodiments of the invention allow for highly multiplexed analysis of fine T cell subsets. These T cell subsets can be identified and selected in the disclosed methods by constellations of expressed cytokines or nuclear transcription factors or the like or combinations thereof. Following this step of the method in the working embodiments disclosed herein, polynucleotides encoding Vα and Vβ T cell receptor polypeptides were then obtained from the selected one or more cells. Typically, polynucleotides encoding Vα and Vβ T cell receptor polypeptides are obtained from a single cell, or a plurality of cells, using a polymerase chain reaction process. Using such methods, we are able to generate cDNA sequences covering the variable regions of the TCR, which then allows reconstruction of the full length heterologous TCRs for use in gene therapy.

Various cytokine secretion inhibitors such as Brefeldin A and/or Monesin can be used in embodiments of the invention. For a description of such secretion inhibitors, see, e.g. Miguel et al., J Immunol Methods. 2012 Oct. 31; 384(1-2): 191-195. In addition, cytokines observed in the methods of the invention can include TNFα and/or IFNγ as well as other cytokines. For a description of cytokines useful in embodiments of the invention, see, e.g., Cox et al., Virology. 2013 Jan. 5; 435(1): 157-169.

In certain embodiments of the invention, T cells used in the methods disclosed herein are obtained from primary peripheral blood mononuclear cells. For example, in some embodiments of the invention, these T cells can be obtained from an individual diagnosed with a pathological condition. In one embodiment of this, T cells can be obtained from an individual diagnosed with a prostate cancer. For example, T cells can be obtained which are selected as those targeting a tissue specific antigen that is expressed in a cancer such as prostatic acid phosphatase (PAP), prostate specific antigen (PSA), prostate-specific membrane antigen (PSMA), Prostate stem cell antigen (PSCA) or the like, the expression of one or more of which persists in prostate cancers such as metastatic prostate adenocarcinoma.

As described herein, the present invention provides methods and materials for making and using modified T cells comprising polynucleotides encoding certain T cell receptor polypeptides. As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta chains. TCRs may exist in alpha/beta and gamma/delta forms, which are structurally similar but have distinct anatomical locations and functions. Each chain is composed of two extracellular domains, a variable and constant domain. Embodiments of the invention include a number of different TCR alpha/beta nucleic acids and their encoded polypeptides.

In typical embodiments of the invention, the polynucleotides encode amino acids of the antigen recognition sequences and further encode additional amino acids such as a constant region of an alpha and/or beta polypeptide, a TM domain, a short cytoplasmic tail, or the like. In illustrative embodiments of the invention, the composition comprises a polynucleotide encoding a TCR Vα polypeptide in combination with a polynucleotide encoding a TCR Vβ polypeptide, wherein such polynucleotides are disposed within one or more vectors such that a Vα/Vβ TCR can be expressed on the surface of a mammalian cell (e.g. a CD8+ T cell) transduced with the vector(s), with this expressed heterologous Vα/Vβ TCR recognizing a peptide associated with a human leukocyte antigen.

Embodiments of the invention include compositions of matter comprising one or more vectors comprising the TCR polynucleotides disclosed herein. A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Typically, the vector is an expression vector. The term “expression” as used herein is defined as the transcription and/or translation of a particular nucleotide sequence driven by its promoter. In this context, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

Typically, a composition of the invention comprises one or more Vα/Vβ polynucleotides, for example a polynucleotide encoding a TCR Vα polypeptide in combination with a polynucleotide encoding a TCR Vo polypeptide such that a Vα/Vβ TCR can be expressed on the surface of a mammalian cell (e.g. a CD8⁺ T cell) transduced with the vector(s), wherein the Vα/Vβ TCR recognizes a peptide associated with a HLA. The term “transduced” or “transfected” or “transformed” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A variety of different antigens can be used in the above-noted TCR embodiments of the invention. For example, in some embodiments of the invention, the antigen is associated with a human leukocyte antigen on an antigen presenting cell, or is disposed within another antigen presenting system or the like (see, e.g. U.S. Patent Publications 20030170212, 20070031442 and 20170283810). In certain embodiments, the antigen comprises a plurality of different antigens (e.g. a plurality of different antigenic peptides). Optionally for example, the antigen comprises a peptide-MHC tetramer.

The technology in this area is reasonably developed and a number of methods and materials know in this art can be adapted for use with the invention disclosed herein. Illustrative methods and materials are disclosed, for example in U.S. Patent Publication Nos. 20190247432, 20190119350, 20190002523, 20190002522, 20180371050, 20180245242, 20180057560, 20170029483, 20170066827, 20160024174, and 20150141347, the contents of which are incorporated by reference.

Further aspects and embodiments of the invention are provided below.

### EXAMPLES

### Example 1: Methods to Clone Human T Cell Receptors from Single Lymphocytes Based on Functional Profiling

TCR Construct Overexpression Allows for TCR mRNA Sequencing Post Intracellular Staining

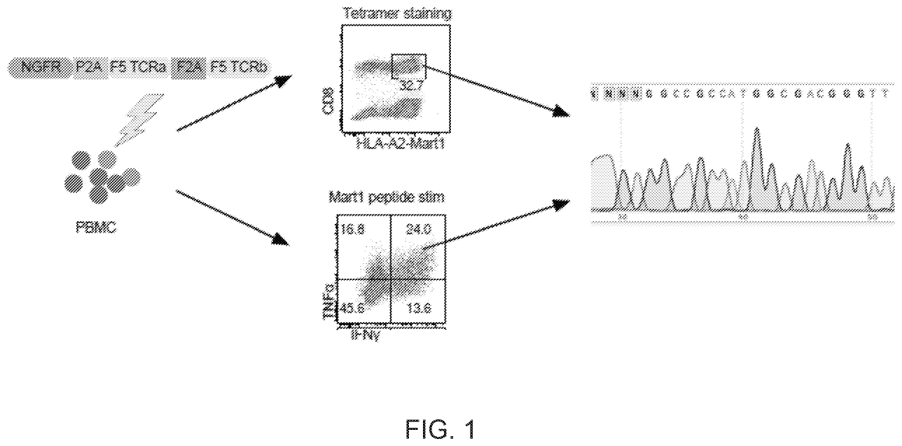

We tested whether the published protocol to analyze mRNA in fixed and permeabilized cells is applicable to TCR biology (9). We induced constitutive expression of high amount of TCR mRNA via retroviral overexpression of F5 TCR (Mart1 specific) in normal PBMC (11). This insured that the biological fluctuation of the target mRNA is kept constant and overall amount is high (FIG. 1). We then stained these cells with Mart1 tetramer as well as activated with cognate peptide pulsing and stained for intracellular TNFα and IFNγ. We sorted both tetramer positive and TNFα/IFNγ double positive cells into separate 96 well plates, for further cloning analysis. We used TCR multiplex primers to run the Reverse Transcriptase-PCR and generate TCR cDNA (FIG. 1). Intracellular staining sample showed bands of the right size and similar quality to tetramer sorted cells, indicating the technique did not significantly decrease the quality of full length TCR mRNA. The bands were further confirmed by sequencing to be F5 TCR. This proof-of-concept experiment showed that when TCR mRNA is abundant, we are able to generate single-cell and paired TCR sequences.

### TCR Alpha/Beta Pairs can be Recovered in Primary T Cells Post Intracellular Staining

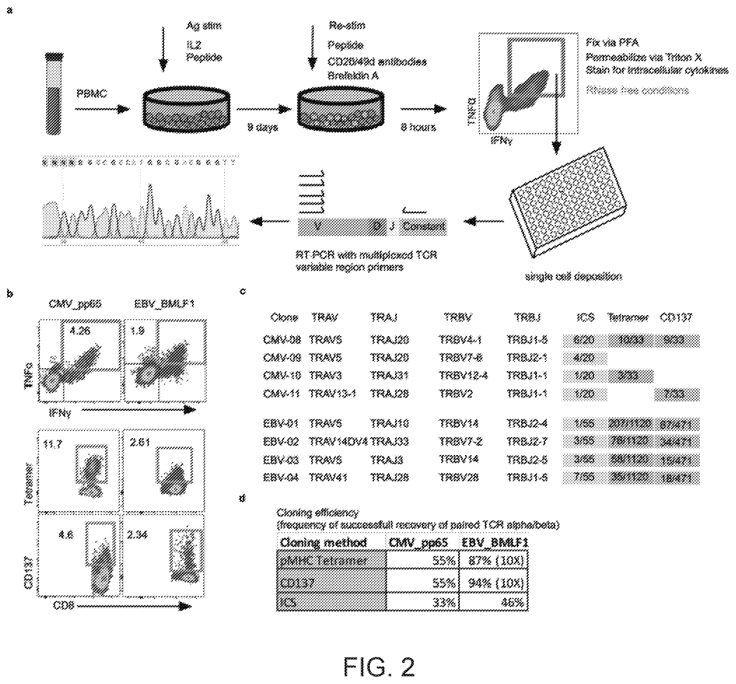

We then further adapted our new protocol to sequence TCR alpha and beta chains in primary PBMCs. We used donor PBMC samples that had high responses to cytomegalovirus (CMV) and Epstein Barr Virus (EBV) antigens. Two different donors are used, one with a CMV response and the other had an EBV response. CMV and EBV are common herpes viruses that infect over 50% of people and generate large memory T cell responses. Therefore, groups developing human T cell cloning techniques often take advantage of responses to these viruses (12). We used a technique we previously described to culture these PBMCs for 9 days in the presence of peptide and then re-stimulate for intracellular staining (FIG. 2a) (13). We stimulated these PBMCs with well described 9mer HLA-A2 CMV pp65 or EBV BMLF1 peptides and subsequently performed ICS for TNFα/IFNγ (FIG. 2b). As a control for sequencing and cloning, we also sorted cells based on CD137 production after stimulation with the cognate peptide and cognate tetramer staining (FIG. 2b). For all three techniques cells were sorted for single-cell sequencing and cloning. CMV response was oligoclonal, with some clones being clearly dominant (10/33 cells are one clone for CMV tetramer staining). The EBV response was quite polyclonal, which is typical of EBV responses. We then compared dominant clones recovered by all three techniques to compare their frequencies within methods (FIG. 2c). The dominant CMV clone was recovered by all three techniques. The dominant EBV clone by tetramer and CD137 staining appeared only once in ICS, however the subdominant clones appeared 3 and 7 times in ICS. This inconsistency in clonal hierarchy may be happening because the best tetramer binder is likely a very high affinity clone, which makes it prone to T cell exhaustion. Exhausted T cell clones lose the ability for polyfunctional cytokine production. Hence these clones would not be abundant in TCRs detected by ICS. Cumulatively, this data shows that TCRs can be sequenced in single cells selected based on ICS. Further, alternative TCR immunodominance hierarchies may be uncovered if ICS is used for TCR selection. This experiment also showed that ICS can be used for TCR cloning with reasonable efficiency, 33-46% compared to 55-94% with live cells (FIG. 2d).

Intracellular Staining for TNFα and IFNγ Identified Antigen Specific TCRs from Primary T Cells.

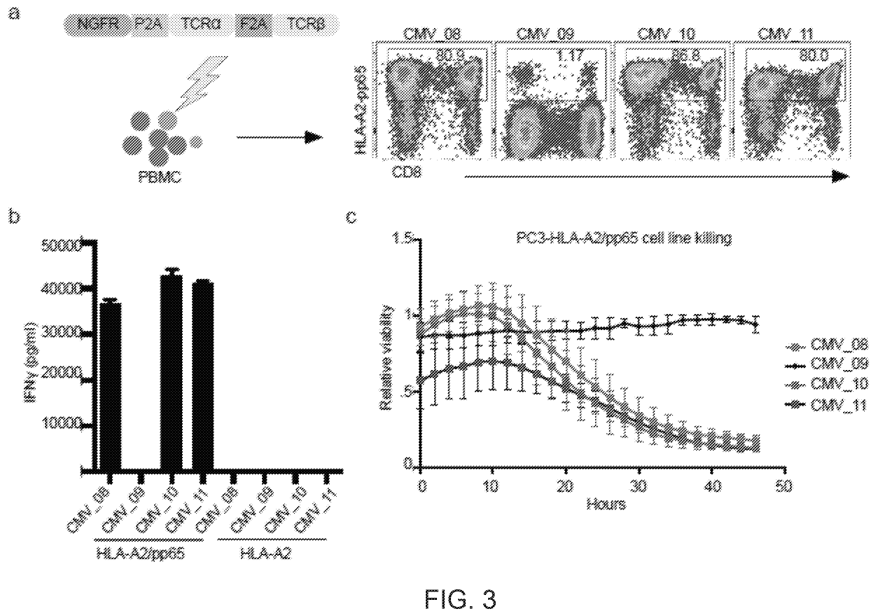

We picked four CMV clones that appeared more than once in the analysis to validate that these are antigen specific clones (FIG. 2c). We cloned these four TCRs into retroviral vectors and overexpressed in healthy PBMC (FIG. 3a). Three of the four highly stained with the CMV tetramer (FIG. 3a). We then proceeded to evaluate functional capability of these clones. We constructed a cell line (PC3, epithelial cancer cell line) to express HLA-A2 and pp65 protein (14). Subsequently, we used this cell line to stimulate the CMV TCR clones and analyze effector function. We co-cultured T cell transduced with CMV TCRs with the PC3 cell line either with or without the pp65. Three of the four CMV TCRs analyzed produced IFNγ in the supernatant during the cu-culture (FIG. 3b). The HLA-A2 construct used was co-linked to GFP such that we were able to monitor viability via IncuCyte. All three of tetramer binding TCRs were able to kill a target cell line expressing full length CMV pp65 protein (FIG. 3c). Therefore, we considered these TCRs to be fully reactive. This further provides evidence that ICS is at least as good as current techniques for identification of TCR alpha beta pairs.

### Methods:

### T Cell Cultures:

Peripheral blood mononuclear cells (PBMCs) from normal donors were used for most experiments, as previously described (13). For the analysis of the Cytomegalovirus (CMV) response we used leukapheresis product from a melanoma patient that was fortuitously available at UCLA. For PBMC culture for ICS analysis we followed the protocol described in our previous publication (13). TCRPMI is used in these experiments and contains 1640 RPMI supplemented with 10% FBS, 1× Glutamax, 1× sodium pyruvate, 10 mM HEPEPS, 1× non-essential amino acids and 50 μM β-mercaptoethanol. Briefly cells were thawed into warm media and rested overnight at 10 e6 million/ml in 24 well plate in TCRPMI media. Subsequently, cells are washed once and resuspended in TCRPMI supplemented with 1 ug/ml of antigenic peptide and 25 U/ml IL2 (peprotech). Half of media is replaced every 2-3 days. On day 9 of peptide and IL2 culture cells are washed 2 times with PBS and once with TCRPMI and resuspended in TCRPMI at 500,000 cells/100 ul of media in 96 well plate for 12-hour rest prior to ICS stimulation.

### Intracellular Staining:

Post 12 hour rest, 100 ul of TCRPMI with 20 ug/ml of antigenic peptide and 2 ug/ml of CD28/49d antibodies (BD) are added in 100 ul of TCRPMI media to each well. Cells are incubated for one hour at 37 C 5% CO₂ and 20 ul of 10× brefeldin A (Biologened) is added to each well. Cells are further incubated for 8 hours. For most analysis cells are stained immediately under RNAse free conditions with a protocol adapted from (Thomsen et al., Fixed single-cell transcriptomic characterization of human radial glial diversity. Nat. Methods 13, 87 (2016)). Most significant deviation from the described protocol is we used 1:400 dilution of RNAsin Plus (Promega) for primary cells. Briefly each well is washed twice in 200 ul of wash buffer: 1% BSA buffer, which contains nuclease free water, 10× molecular biology grade PBS, 1% nuclease free BSA (Gemini), and 1:400 RNAsin Plus (Promega). Then surface antibodies resuspended in 100 ul of wash buffer are added to each well. Surface antibodies include: CD3-APCCy7 (eBio), CD8a-PE, CD4-PECy7. After staining for 15 minutes at 4 C cells are washed with wash buffer and fixed with 100 ul of 4% paraformaldehyde (PFA) for 10 minutes at 4 C. Then cells are washed twice with 200 ul of wash buffer/well and resuspended in wash buffer with 0.1% Triton X-100 (Sigma) for 10 minutes. Cells are then washed with 200 ul per well of wash buffer and intracellular antibodies are added in 100 ul of wash buffer for 20 min. Intracellular cytokine antibodies used are IFNγ-APC and TNFα-FITC. Cells are then washed and resuspended in wash buffer for FACS analysis.

### FACS Single-Cell Deposition, RT-PCR Analysis, and TCR Cloning:

A protocol for our TCR cloning strategy is described in detail (13, 15). Briefly, cells are gated on lymphocytes by light scatter, single events, CD3+, CD8+, TNFα+/IFNγ+ or tetramer/CD137+. With live cell analysis, dapi was also added to the gating hierarchy to make sure detection of live cells. Background signal is either set on DMSO stimulation or irrelevant tetramer, to maximize detection of true positive events. Antigen specific T cells are deposited at 1 cell/well into 96 well plate containing lysis buffer. Plates are immediately placed on dry ice and then frozen at −80 C for further analysis. Subsequently, plates are thawed on ice and incubated at 56 C for 1 hour. This ensures reverse cross linking of mRNA from protein, to allow the RT-PCR reaction. Each cell is then split into two wells to allow independent sequencing of alpha and beta TCR chains. RT-PCR reaction is performed with multiplex TCR variable region primers and alpha and beta constant region primers using Qiagen one step RT-PCR to generate TCR cDNA. Nested alpha and beta chain PCR is performed to amplify the TCR cDNA and the product is then sanger sequenced (Laragen). Assembly PCR and restriction enzyme cloning is performed to generate the retroviral constructs, per the following map: tNGFR-P2A-TCRα-F2A-TCRγ.

### Retroviral Transduction and TCR Functional Assays:

Normal donor PBMCs obtained from University of California Los Angeles CFAR core are thawed and stimulated with human CD3/28 beads (Thermo) at 1:1 ratio in Aim V media with Human AB serum, 50 U/ml IL2, Glutamax and 50 μM β-mercaptoethanol. Stimulation is done in 24 well TC plate at 1 e6/ml and 2 ml/well. After 2 days about 1.5 ml of media is removed from each well and 1 ml of retroviral supernatant is added with 5 ug/ml polybrene. Cells are then centrifuged at 1350 G for 90 minutes at 30 C to increase mediate transduction. Post transduction about 1 ml is removed from each well and 1 ml of T cell media is added with 50 U/ml IL2. Next day viral transduction via centrifugation is repeated. The following day cells are washed once with T cell media and each well is resuspended in 2 ml of T cell media. Our TCR constructs contain the murine constant region. So TCR export to the cell surface was evaluated by TCR murine beta chain FACS staining.

### TCR Coculture for ELISA and Killing Assays:

We followed a previously published protocol for TCR functional validation (13). Briefly, TCR transduced PBMCs were cocultured with target cell line PC3 that expressed HLA-A2 and pp65 CMV protein. Cocultures were set up at 2:1 E:T ratio in 100 ul of F12K media supplemented with 10% FBS and L-glutamine in 96 well, flat bottom plate. Cell killing was visualized using the IncuCyte system (Sartorius), which quantified GFP levels in PC3 cells. At 48 hours 50 ul of supernatant was collected and IFNγ ELISA was performed (BD).

### REFERENCES LISTED IN TEXT ABOVE

1. Feigal E G, DeWitt N D, Cantilena C. Peck C. Stroncek D. At the end of the beginning: immunotherapies as living drugs. Nature Immunology. 2019; 20(8):955-62.
2. Rodenko B, Toebes M. Hadrup S R, van Esch W J E, Molenaar A M, Schumacher T N M, et al. Generation of peptide-MHC class I complexes through U V-mediated ligand exchange. Nature Protocols. 2006:1(3):1120-32.
3. Wolfl M, Kuball J, Ho W Y, Nguyen H, Manley T J, Bleakley M. et al.
Activation-induced expression of CD137 permits detection, isolation, and expansion of the full repertoire of CD8+ T cells responding to antigen without requiring knowledge of epitope specificities. Blood. 2007; 110(1):201-10.
4. Betts M R, Brenchley J M, Price D A, De Rosa S C, Douek D C, Roederer M, et al. Sensitive and viable identification of antigen-specific CD8+ T cells by a flow cytometric assay for degranulation. Journal of immunological methods. 2003:281(1-2):65-78.
5. Slifka M K, Rodriguez F, Whitton J L. Rapid on/off cycling of cytokine production by virus-specific CD8+ T cells. Nature. 1999; 401(6748):76.
6. Freeman B E, Hammarlund E, Raué H-P, Slifka M K. Regulation of innate CD8+ T-cell activation mediated by cytokines. Proceedings of the National Academy of Sciences. 2012; 109(25):9971-6.
7. Linnemann C, van Buuren M M, Bies L. Verdegaal E M E, Schotte R. Calis J J A, et al. High-throughput epitope discovery reveals frequent recognition of neo-antigens by CD4+ T cells in human melanoma. Nature Medicine. 2014:21:81.
8. Waldrop S L, Pitcher C J, Peterson D M, Maino V C, Picker L J. Determination of antigen-specific memory/effector CD4+ T cell frequencies by flow cytometry: evidence for a novel, antigen-specific homeostatic mechanism in HIV-associated immunodeficiency. The Journal of clinical investigation. 1997; 99(7):1739-50.
9. Thomsen E R, Mich J K, Yao Z, Hodge R D, Doyle A M, Jang S, et al. Fixed single-cell transcriptomic characterization of human radial glial diversity. Nature methods. 2016; 13(1):87.
10. WO 2016/161023 to Jang et al.
11. Johnson L A, Heemskerk B, Powell D J, Cohen C J, Morgan R A, Dudley M E, et al. Gene transfer of tumor-reactive TCR confers both high avidity and tumor reactivity to nonreactive peripheral blood mononuclear cells and tumor-infiltrating lymphocytes. The Journal of Immunology. 2006; 177(9):6548-59.
12. Klenerman P, Oxenius A. T cell responses to cytomegalovirus. Nature Reviews Immunology. 2016:16(6):367.
13. Bethune M T, Li X-H, Yu J, McLaughlin J, Cheng D, Mathis C, et al. Isolation and characterization of N Y-ESO-1-specific T cell receptors restricted on various MHC molecules. Proceedings of the National Academy of Sciences.
2018:115(45):E 10702-E 11.
14. Kaighn M, Narayan K S, Ohnuki Y, Lechner J F, Jones L. Establishment and characterization of a human prostatic carcinoma cell line (PC-3). Investigative urology. 1979; 17(1):16-23.
15. Smith D J, Liu S, Ji S, Li B, McLaughlin J, Cheng D, et al. Genetic engineering of hematopoietic stem cells to generate invariant natural killer T cells. Proceedings of the National Academy of Sciences. 2015; 112(5):1523-8.

### Example 2: Methods for Sequencing mRNA in Single Cells in Parallel with Quantification of Intracellular Phenotype

As disclosed in the text above, we showed how T Cell receptor (TCR) alpha and beta chains can be sequenced from antigen specific T cells selected on cytokine production. In this Example, we report significant advances in this technology that allow for global mRNA profiling of cells selected on an intracellular phenotype by high throughput, droplet-based single cell sequencing technologies. Such droplet-based single cell sequencing technologies are described, for example, in Macosko et al., CELL Volume 161, Issue 5, Pages 949-1230 (21 May 2015), Salomon et al., LAB ON A CHIP, 2019, 19, 1706, and U.S. Patent Publication Nos. 20190127782, 20200108393, and 20200115753, the contents of which are incorporated herein by reference.

Briefly, in this Example, we modified earlier described protocols by designing a method of cell permeabilization that uses a cleavable crosslinker. This allows for parallel intracellular protein and mRNA profiling in single cells by droplet-based technologies such as the one offered by 10× Genomics. The advances we describe in this Example allow for high throughput parallel analysis to be done for intracellular proteins and is of high value through out cell biology. Such protocols are immediately applicable to single cell multimodal omics analysis.

Illustrative embodiments of the invention are adapted for sequencing of TCRs in Regulatory T cells. Treg cells are exclusively identified by their expression of the FOXP3 nuclear transcription factor (see, e.g. Wakamatsu et al., Biochem Biophys Res Commun. 2018 Sep. 18; 503(4):2597-2602; and UniProtKB—Q9BZS1 (FOXP3_HUMAN)). However, direct TCR analysis has not been done in human cells because mRNA would be degraded. As discussed below, we showed that we can identify full length Treg TCRs using the methods disclosed herein. Additional details are discussed in EXAMPLE 3 below.

### Methods Using Non Chemically Cleavable Crosslinkers

For comparative studies, methods using a non-chemically cleavable crosslinker to crosslink cellular polypeptides (and then manually clone cells etc. etc.) was performed. In these methods, cells were deposited into single wells by FACS following crosslinking by the non-chemically cleavable crosslinker. In contrast to the “CLint-Seq” methodologies that disclosed herein, this technique, one that uses a non-chemically cleavable crosslinker was highly laborious, and less green. Moreover, this methodology failed to work with, or be adaptable to, 10× Genomics droplet-based single cell sequencing platforms known in the art. These comparative non-chemically cleavable crosslinker methods in this technology are briefly discussed below.

### Intracellular Staining Via PFA Crosslinking:

For TCR sequencing cells can be stained immediately under RNAse free conditions using the adaptation of the FRISCR protocol. Each well in a plate is washed twice with 200 μL of wash buffer, which contains nuclease free water (Thermo Fisher, cat. no. 4387936), 10× molecular biology grade PBS, 1% nuclease free BSA (Gemini cat. no. 700-106P)), and 1:400 RNAsin Plus (Promega cat. no. N2615). The cells are stained with surface antibodies such as: CD3-APCCy7 (Thermo Fisher, cat. no. 47-0036-42), CD8a-PE (Thermo Fisher, cat. no. 12-0088-42), CD4-PECy7 (Biolegend, cat. no. 300512). After staining for 15 minutes at 4 C cells are washed with wash buffer and fixed with 100 μL of 4% PFA (EMS cat. no. 15710) for 10 minutes on ice. Then cells are washed twice and resuspended in 1% BSA buffer with 0.1% Triton X-100 (Sigma-Aldrich cat. no. T8787) for 10 minutes. Cells are then washed and subsequently stained with intracellular antibodies in wash buffer for IFNγ-APC (Biolegend cat. no. 506510), TNFα-FITC (Biolegend cat. no. 502906). FOXP3-A488 (Biolegend cat. no. 320012), msIgG1-A488 (Thermo Fisher, cat. no. MG120). Cells are then washed and resuspended in wash buffer for FACS analysis.

TCR Cloning Method Using FACS Deposition of Single Cells into PCR Wells.

PBMCs were gated on live lymphocytes by light scatter, single events, CD3⁺, CD8⁺, TNFα⁺/IFNγ⁺ or tetramer/CD137⁺. Antigen specific T cells were deposited at 1 cell/well into 96 well PCR plate containing 10 mM Tris pH 8.0 RNAsin 1:40 dilution (Promega cat. no. N2515). Plates were flash frozen and kept at −80 C for further analysis. Subsequently, plates were thawed on ice and incubated at 56 C for 1 hour to reverse mRNA-protein crosslinking. Each well was then split into two for independent sequencing of alpha and beta TCR chains. RT PCR reaction was performed with multiplex TCR variable region primers (IDT) and alpha and beta constant region primers using Qiagen one step RT-PCR (Qiagen cat. no. 210212) to generate TCR cDNA. Nested alpha and beta chain PCR was performed to amplify the TCR cDNA and the product was then sanger sequenced (Laragen Inc). Assembly PCR and restriction enzyme cloning was performed to generate the retroviral constructs, per the following map: tNGFR-P2A-TCRα-F2A-TCRβ.

### Clint-SEQ Embodiment

In this embodiment, we sought to develop new methods which are, for example, specifically designed to be compatible with droplet-based single cell sequencing techniques, a state-of-the-art method that allows for sequencing mRNA in thousands of cells.

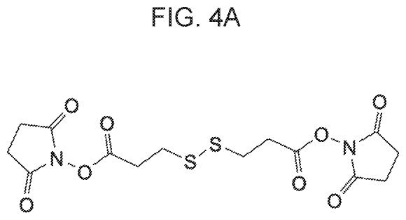

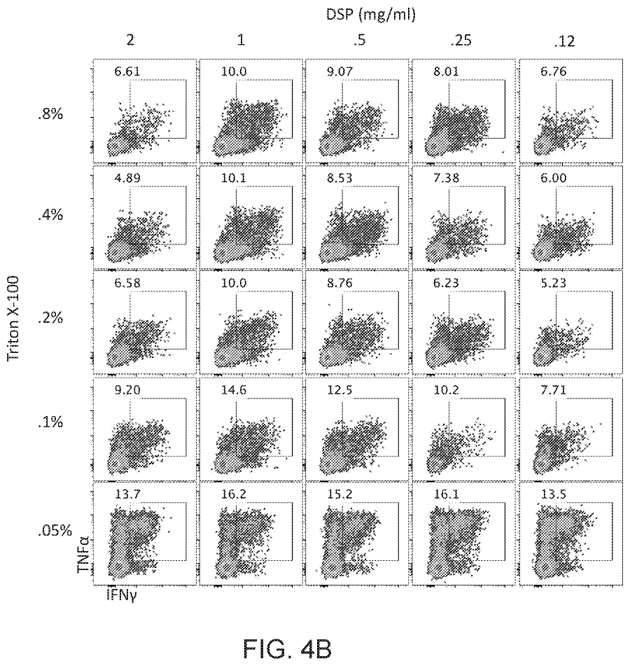

In order to overcome difficulties in this art, we developed a new methodology, termed “CLint-Seq”, that uses a chemically cleavable crosslinker, for example DSP (Lomant's Reagent, 3,3′-Dithiodipropionic acid di(Nhydroxysuccinimide ester)), to fix cells (FIG. 4). DSP reacts with primary amines and has a sulfide bond in the center, which can be reduced with a reducing agent. This property allows mRNA to be tethered to protein and then be released under reducing conditions. DSP has previously been used to preserve cells prior to single cell mRNA sequencing using the Fluidigm C1 machine¹. However, we could not predict the effects of using chemically cleavable crosslinkers (and the associated method steps specific to these crosslinkers) could be successfully used to permeabilize cells in the intracellular staining of cells in suspension and the further method steps that are typically performed on such cells. Surprisingly these “CLint-Seq methods work very well, and for example, allowed for efficient TCR alpha/beta pair mRNA recovery via a droplet-based single cell sequencing platform from cells that have been fixed and permeabilized by our new methods.

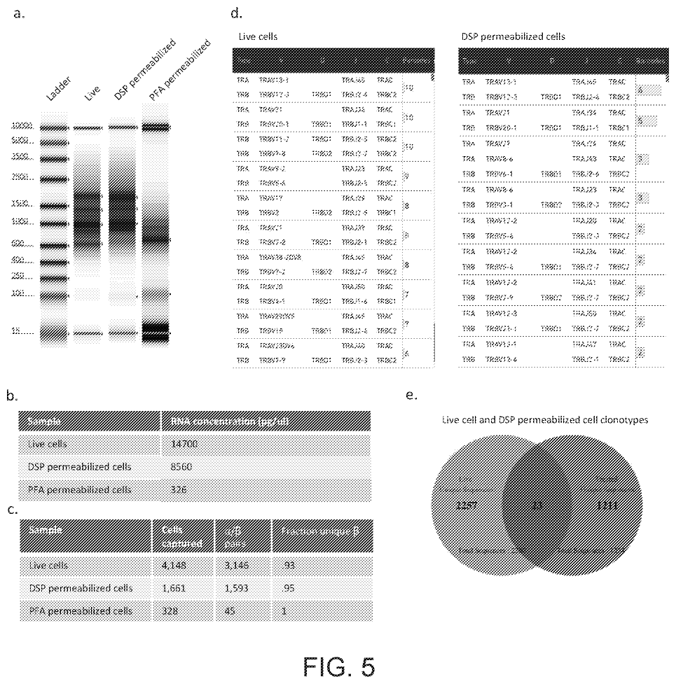

In one working embodiment of our methodology, we first determined the range of DSP as well as Triton X-100 permeabilization that would allow for sensitive detection of cytokines in stimulated T cells. We determined that 1-0.25 mg/ml of DSP and 0.1%-0.05% of Triton X-100 is compatible with cytokine staining (FIG. 4). Next we subjected a mixed population of human T cells to crosslinking with 0.25 mg/ml DSP and permeabilization with 0.05% Triton X-100. These cells were then submitted for human V(D)J 10× Genomics library construction and subsequent next generation sequencing. At the library construction step, we compared concentration of cDNA post PCR enrichment. Live and DSP treated cells showed comparable quality of cDNA, 14700 pg/μL and 8560 pg/μL respectively (FIG. 5). While, PFA treated cells had poor quality of cDNA generation, 326 pg/μL. Post sequencing data was analyzed per the 10× genomics pipeline. Again, live and DSP permeabilized cells returned similar results, 3,146 and 1,593 alpha/beta pairs respectively. While, the cells permeabilized using PFA returned only 45 pairs, illustrating extremely poor recovery. We also determined that DSP does not decrease the fidelity of cDNA synthesis, as live and DSP treated cells showed exact same nucleotide sequences. These T cells are not specific for a particular antigen, thus only 23 clonotypes were shared between live and DSP treated cells (FIG. 5). However, the fact that some clonotype are shared provides evidence of faithful recovery of TCR sequences.

Single Cell mRNA Sequencing in Cells with Specific Intracellular Phenotypes:

Global mRNA sequencing at the single cell level has revolutionized cell biology. We can unbiasedly investigate what is happening to a particular cell, which helped define new phenotypes and drug targets. However, proteins are the functional units of cells. Therefore, mRNA sequencing has major limitations. For example, some important proteins with long half-lives will have very low mRNA abundance (2). Thus, it is difficult to use mRNA sequencing to detect such proteins and consequently define cell phenotypes.

As disclosed herein, CLint-Seq can be used sequence global mRNA profiles in cells selected for expression of multiple intracellular proteins or for parallel mRNA and protein sequencing in single cells. For example, any cell type can be stained intracellularly for multiple proteins and cells can be sorted by FACS for a desired phenotype and subsequent single cell sequencing. Alternatively. mRNA and protein can be quantified in parallel by staining cells with antibody-oligo complexes. Techniques such as CITE-seq and REAP-seq allow for simultaneous detection of protein and mRNA in single cells using oligo tagged antibodies and subsequent single cell sequencing (3,4). However, this analysis is limited to surface proteins as the assumption is that mRNA would become degraded upon intracellular staining with Antibody-oligo constructs. Our method of cell crosslinking using compounds such as DSP and permeabilization allows for massive parallel protein and mRNA sequencing, without the limitation to surface proteome.

These techniques are made available to the broad scientific community through the sale of easy to use kits by companies such as BD, Biolegend and Thermo Fisher Scientific. Biolegend commercialized CITE-seq and REAP-seq in a series of reagents called TotalSeq. Therefore, the technical knowledge employed in CLint-Seq would be of direct interest to these companies.

### Application to Personalized T Cell Therapy Engineering:

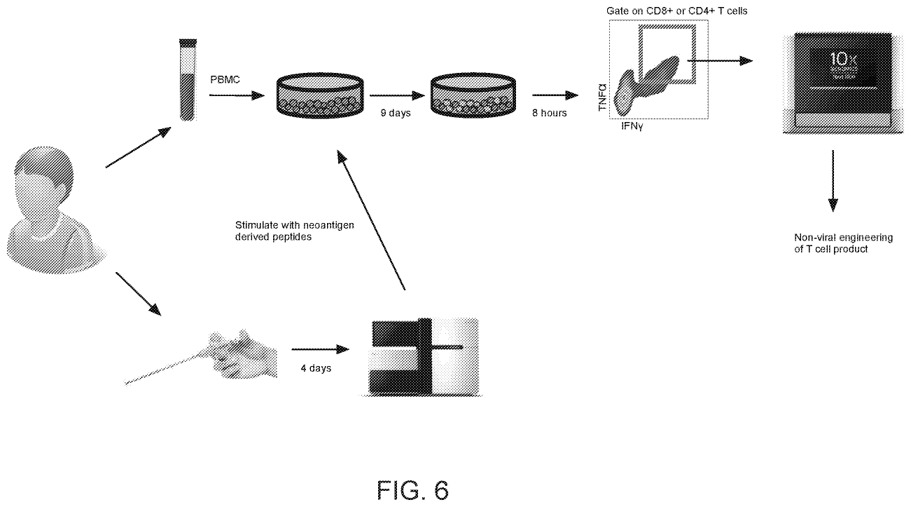

One use of CLint-Seq as disclosed herein is to quickly generate large numbers of antigen specific TCRs for both class I and class II restricted antigens. This is of direct interest to the field of personalized Immuno-Oncology (10) (FIG. 6). We envision we can obtain a blood sample and needle biopsy from a patient with cancer and subsequently use next generation sequencing to map neoantigens. Peptides can then be tiled through these regions for stimulation of patient PBMCs. Reactive CD4⁺ and CD8⁺ T cells will be identified by cytokine production and submitted for 10× genomics sequencing. TCR affinity can be enhanced by including markers of affinity such as Nur77 at T cell selection step⁵. Subsequently, TCR alpha/beta pairs will be synthesized and non-virally introduced into autologous or allogeneic T cells. Autologous PBMCs can be sourced at the biopsy timepoint and cryopreserved for 20 days. We estimate that the whole process will take about 28 days, but we can think about optimizing it to make it as little as three weeks.

A similar approach is currently pursued by PACT pharma. PACT uses algorithm prediction to design class I MHC tetramers which are then used to capture antigen specific CD8⁺ T cells.

The CLint-Seq approach will have higher sensitivity and identify a larger number of tumor reactive TCRs in more patients. Epitope prediction algorithms are infamous for predicting a large number of false-positive epitopes as well as missing real epitopes. Therefore, an approach that relies on overlapping peptides across a neoantigen region will identify higher proportion of positives. Further, MHC class I tetramers work well for CD8⁺ T cells, however, it is very difficult to construct MHC class II tetramers and once made they require very high affinity to detect a CD4⁺ T cell (6). Therefore, tetramer-based detection of tumor reactive CD4⁺ T cells is difficult. However, CD4⁺ T cells represent an important component of the cytotoxic T cell response and have been shown to direct antitumor responses in multiple models (7). In addition, CD4⁺ T cells are two-fold more abundant than CD8⁺ T cells and the tolerance mechanisms are more promiscuous for CD4⁺ T cells which results in a lower rate of deletion. Thus, having the ability to capture antigen reactive CD4⁺ T cells will result in greater number of antigen reactive TCRs per patient as well as identify higher number of patients that are suitable for personalized therapy engineering.

### TCR Tuning Based on Transcription Factor Phenotype:

T cell function and performance can be inferred from the expression of single or multiple transcription factors (TF). Parallel TF and TCR profiling has not been possible in the past. We already illustrated this approach by sequencing TCRs in Treg cells, however there are many other TFs that can be profiled to select TCRs of desired specificity and function. TF phenotyping can be included in the pipeline we describe for IO to enhance ability to engineer durable T cell responses. We present a table that summarizes which TCRs can be selected based on presence/absence of common TFs (Table 1).

TABLE 1

| TCR performance | FOXP3 | Helios | Nur77 | NFAT |
|---|---|---|---|---|
| High affinity | | | + | |
| TCR | | | | |
| CD4⁺, | +/− | − | + | + |
| neoantigen | | | | |
| reactive | | | | |
| CD4⁺, | | | + | + |
| cytotoxic | | | | |
| Autoimmune | + | + | + | |
| pathology | | | | |

### Methods:

### Cultures:

All lymphocyte cultures are performed as described above and in EXAMPLE 3.

### Cell Fixation and Permeabilization:

All buffers except crosslinker step contain 1:400 RNAsin (Promega) and molecular biology grade PBS to inhibit RNA degradation. Experiments described in this here did not include antibody, however all incubations were performed as if antibodies were included to recapitulate conditions of intracellular staining. For these experiments we used a mixture of human activated PBMCs that contained 20% Jurkat cells. Cells were first washed in 1% BSA (Gemini) buffer with 1:400 RNAsin (wash buffer) and incubated for 15 minutes on ice. DSP is stored at −20 C in a desiccant filled container. Immediately prior to experiment DSP is left at room temperature for at least 30 minutes and then prepared to a concentration of 50 mg/ml in molecular biology grade DMSO (Sigma). Then 1 mg/ml solution is prepared in molecular biology grade PBS, by vortexing 20 ul of DSP in a 15 ml conical tube and adding 1 mL of PBS with P1000. DSP is filtered using a 40 μm Flowmi strainer (Sigma). Then 0.25 mg/ml solution is prepared. Then cells are washed twice with PBS and resuspended in 200 μL of 0.25 mg/ml DSP (Thermo Fisher). Cells are incubated at room temperature for 30 minutes and quenched with 200 mM Tris (Thermo Fisher). Cells are then washed and incubated for 10 minutes with 100 μL of 0.05% Triton X-100 (Thermo Fisher) in wash buffer. Subsequently, cells are washed and resuspended in wash buffer for 20 minutes. Then, cells are washed again and resuspended at 700 cells/μL in 0.04% BSA with 1:400 RNAsin to be submitted for 10× library preparation.

### 10× Library Preparation:

10× genomics human V(D)J libraries were prepared by the UCLA Technology Center for Genomics & Bioinformatics per the typical 10× genomics library construction protocol without any modifications to this protocol.

### 10× Library Sequencing:

Single cell TCR libraries were sequenced by Illumina NextSeq. Data was analyzed by using 10× genomics pipeline to generate Vloupe files.

### Example 2 References

1. Attar, M. et al. A practical solution for preserving single cells for RNA sequencing. Scientific Reports 8, 1-10 (2018).
2. Schwanhausser, B. et al. Global quantification of mammalian gene expression control. (2013) doi:10.1038/nature10098.
3. Stoeckius, M. et al. Simultaneous epitope and transcriptome measurement in single cells. Nature Methods 14, 865-868 (2017).
4. Peterson. V. M. et al. Multiplexed quantification of proteins and transcripts in single cells. Nature Biotechnology 35, 936-939 (2017).
5. Ashouri, J. F. & Weiss, A. B Cells Antigen Receptor Signaling in Human T and Endogenous Nur77 Is a Specific Indicator of. (2016) doi:10.4049/jimmunol.1601301.
6. Sabatino, J. J., Huang, J., Zhu, C. & Evavold, B. D. High prevalence of low affinity peptide-MHC II tetramer-negative effectors during polyclonal CD4+ T cell responses. Journal of Experimental Medicine 208, 81-90 (2011).
7. Linnemann, C. et al. High-throughput epitope discovery reveals frequent recognition of neo-antigens by CD4+ T cells in human melanoma. Nature medicine 21, 81 (2014).
8. Thomsen, E. R. et al. Fixed single-cell transcriptomic characterization of human radial glial diversity. Nature methods 13, 87 (2016).
9. Pitcher, C. J. et al. HIV-1-specific CD4+ T cells are detectable in most individuals with active HIV-1 infection, but decline with prolonged viral suppression. Nature medicine 5, 518 (1999).

### Example 3: Antigen-Specific T Cell Receptor Identification by Single-Cell Intracellular Phenotyping

T cell immunotherapeutic pipelines require techniques for robust identification of antigen reactive T cell receptors (TCRs). Conventionally available methods are peptide-MHC multimers and surface activation markers. Peptide-MHC multimers are laborious to construct and are optimized to detect CD8⁺ T cells. Surface activation markers can detect both CD4⁺ and CD8⁺ T cells, however, are not specific because of expression on non-antigen specific T cells. In this example, we describe the use of mRNA sequencing via a methodology that we have developed and termed “Crosslinker regulated intracellular phenotype” (“CLint-Seq”) for efficient recovery of antigen-specific TCRs in cells stained for intracellular proteins such as cytokines or transcription factors. Cytokine staining for TNFα and IFNγ allowed for identification of Cytomegalovirus and Epstein-Barr virus reactive TCRs with efficiency similar to state-of-the-art tetramer methodology. Optimized intracellular staining conditions, that use a chemically reversible primary amine crosslinker DSP, allowed permeabilized cells to undergo single-cell mRNA sequencing in a fluid droplet via the Drop-Seq format. This method enables high-throughput characterization of low frequency TCRs specific for tumor or viral antigens.

Adoptive T-cell transfer is a promising immunotherapeutic modality potentially applicable to many human diseases, including cancers, viral illnesses, and autoimmune disorders (1-3). T cells engineered to express a cancer specific T cell receptor (TCR) can mediate regression of late stage tumors (4). Expanded populations of Cytomegalovirus specific T cells have been used to control viremia (5). A crucial step for translating these advances to other cancers and new viral pathologies is the identification of target proteins and their corresponding TCRs. However, state-of-the-art approaches for TCR characterization identify numerous non-peptide specific TCRs, require a priori knowledge of the epitope, and necessitate new reagents for every new epitope (6-8). Consequently, numerous studies show low recovery of TCR reactivities from predicted neo-antigens (9, 10).

T cell receptors can be sequenced from live cells that have been activated and identified by expression of activation markers such as CD137, CD107a/b or surface-captured secreted cytokines (11-14). Cytokines can be captured as they are being secreted by using an antibody sandwich method (12, 13). It is difficult to rely on these markers for TCR cloning, as they are expressed at low level and non-specifically upregulated (expressed on >1% of CD8⁺ T cells) (14). Unfortunately, tumor associated antigens and the neo-antigen specific T cells of interest are also found at low frequencies (less than 1% of CD8+) even after expansion (9, 10).

Intracellular staining (ICS) is a common immunology technique for enumerating antigen specific T cell responses (15, 16). Cytokine production is antigen dependent and drops once antigen is removed (17). Such control is physiologically required to prevent autoimmune pathologies due to cytokine potency (17). This stringent control makes cytokine production a highly specific marker of T cell activation. Yet, ICS requires fixation which is thought to degrade mRNA. However, TCRs are sequenced from the mRNA to identify the coding sequence in a particular cell. Thus, the field would benefit from a new approach that would couple ICS for detection of antigen specific T cells to TCR sequencing for clone characterization.

Here we describe mRNA sequencing via a methodology we term “Crosslinker regulated intracellular phenotype” (“CLint-Seq”). This methodology allows for mRNA sequencing in parallel with characterizing intracellular phenotype in single cells. This method has been designed to be compatible with droplet-based single-cell sequencing formats such as the one offered by 10× Genomics. We validate the CLint-Seq technology by cloning TCRs against Cytomegalovirus and Epstein-Barr Virus from effector T cells identified by intracellular TNFα and IFNγ. In addition, we characterize human CD4⁺ regulatory T (Treg) cell TCRs by nuclear transcription factor FOXP3 profiling. Therefore, CLint-Seq is shown to be broadly applicable for robustly finding antigen specific TCRs with desired on-target activity.

### Results:

Intracellular Staining Identifies Antigen Specific T Cells with a Lower Rate of False Positives than CD137 Activation Marker

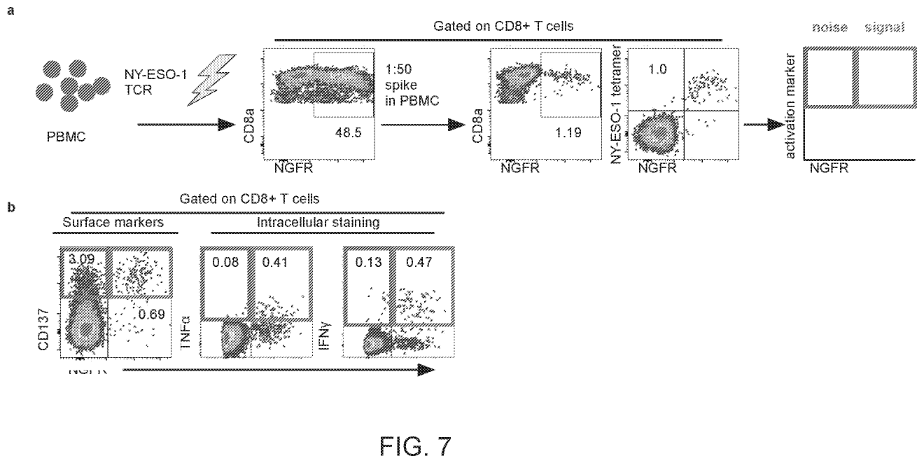

To compare specificity of surface activation markers and ICS for the identification of reactive T cells, we tested the ability of each technique to identify true positive antigen-reactive T-cells in an activation assay. We selected the melanoma antigen NY-ESO-1 and a cognate TCR (clone 1G4) as a model (18). We used a retrovirus construct with 1G4 TCR co-linked to NGFR to estimate the specificity of the ICS approach compared to the CD137 method (18). We defined false positives as events that express an activation marker yet do not carry the cognate TCR. To produce a simulated population that is about 1% positive, a TCR-transduced population was spiked into an untransduced population of cells to create a fixed ratio of positive cells in the culture. After antigen stimulation, flow cytometry was performed for CD137 and compared to ICS for TNFα or IFNγ (FIG. 7a). Both assays were comparable in terms on sensitivity with 0.69% of CD8⁺ T cells in the CD137⁺/NGFR⁺ compartment and 0.41% and 0.47% in the TNFα⁺/NGFR⁺ and IFNγ⁺/NGFR⁺ compartments respectively. However, 3.09% of CD8⁺ T cells were in the NGFR⁻/CD137⁺, illustrating a high background in this assay due to bystander T cell activation (FIG. 7b). But, only 0.08% and 0.13% of CD8⁺ T cells were in the NGFR⁻/TNFα⁺ or NGFR⁻/IFNγ⁺ compartments respectively (FIG. 7b), providing evidence that ICS could provide greater specificity for the detection of true positives.

TCR Alpha/Beta Pairs can be Recovered in Primary T Cells after Intracellular Staining

We induced constitutive expression of a melanoma antigen MART-1 TCR (clone F5) in PBMC to test if TCRs could be sequenced from intracellularly stained T cells (18). This insured that the biological fluctuation of the target mRNA was constant (FIG. 1). We then activated these cells with MART-1 peptide and stained for intracellular TNFα and IFNγ, adapting a published method of single-cell mRNA sequencing in permeabilized cells that used paraformaldehyde (PFA) as a crosslinker (19). In parallel, a control arm was set up where cells were selected with MART-1 tetramer. Reactive cells were then singly deposited by FACS for alpha/beta paired TCR sequencing (FIG. 1). TCR clones were isolated from single-cell RT-PCR reactions. Both techniques had equivalent efficiency, measured as a fraction of TCR alpha/beta pairs recovered (75%). This proof-of-concept experiment revealed single-cell TCR mRNA could be sequenced from cells that were stained for intracellular antigens.

Human T cell immunology field is interested in identifying novel antigen-specific TCRs from populations of human T cells. As a proof-of-concept, we sought to obtain functional TCRs against Cytomegalovirus and Epstein Barr virus from two different donors. Cytomegalovirus and Epstein Barr Virus are common Herpes viruses that infect over 50% of people and generate large memory T cell responses (20). We cultured PBMCs for 9 days in the presence of cognate peptide and re-stimulated them for ICS (FIG. 2a) (18). We subsequently performed ICS and isolated single-cells that expressed TNFα and IFNγ (FIG. 2b). The TCRs of these cells were then sequenced by RT-PCR with multiplex primers. As a control, we also sorted and sequenced cells based on CD137 production and cognate tetramer staining (FIG. 2b).

We then compared TCR sequencing efficiencies between the three techniques (FIG. 2c). Although the CMV response was oligoclonal, some clones were clearly dominant (10/33 cells were a single clone by CMV tetramer staining). The dominant CMV clone was recovered by all three techniques. In contrast, the TCRs obtained from cells exposed to EBV were polyclonal, which is typical of EBV responses. The dominant EBV clone identified by tetramer and CD137 staining appeared only once in intracellular staining, however the subdominant clones appeared 3/55 and 7/55 times in ICS. This inconsistency in clonal hierarchy between ICS and tetramer based cloning staining may be caused by T cell exhaustion. Tetramers preferentially bind highest affinity clones, which are the most prone to T cell exhaustion and lose the ability for polyfunctional cytokine production. Hence, these clones would not be abundant in TCRs detected by ICS. Cumulatively, this data showed that ICS could enable the isolation and sequence of TCRs in single cells. This experiment also showed that ICS can be used for TCR cloning with reasonable efficiency, 33-54% compared to 55-94% with live cells (FIG. 2d).

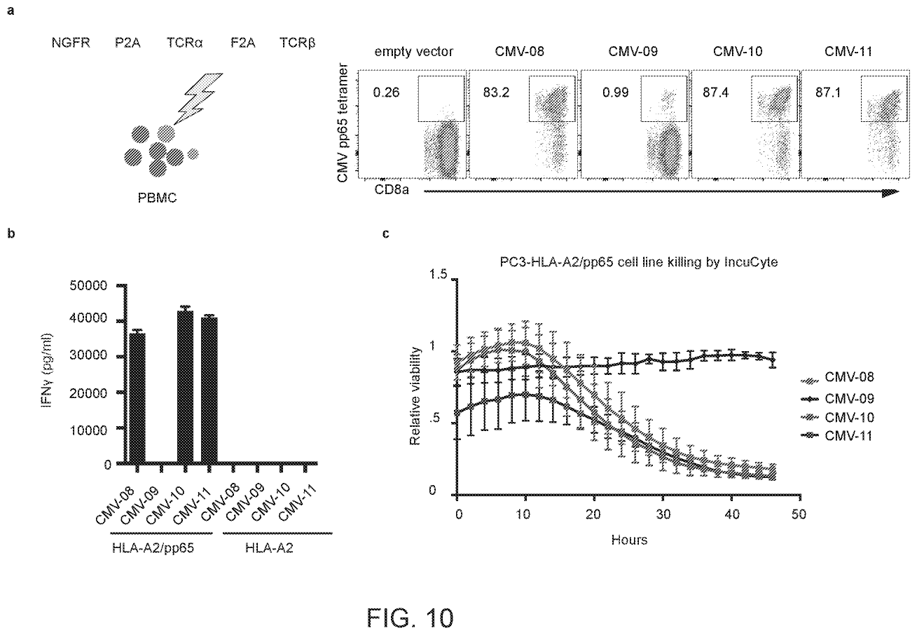

Ultimately, the true test for TCR antigen specificity is clonal sequence isolation and transplant into normal human T cells. This test if an assay will identify high affinity TCRs, rather than simply cross reactive clones. We picked four CMV TCR clones that appeared multiple times in the analysis, cloned them into retroviral vectors and overexpressed them in human PBMCs (FIG. 10a). We then proceeded to evaluate the functional capability of these clones for cytokine production in a cytotoxicity. T cells transduced with the CMV TCRs were cocultured with a PC3 epithelial prostate cancer cell line that expressed HLA-A2 with or without the CMV pp65 protein. All three CMV TCRs that appeared in both tetramer and ICS based selection led to the production of IFNγ (FIG. 10b) when T cells were co-cultured with target cells that express CMV pp65 protein. Additionally, these same T cells were able to specifically kill target cells that expressed the protein (FIG. 10c). These results indicate that TCRs discovered from ICS are fully reactive.

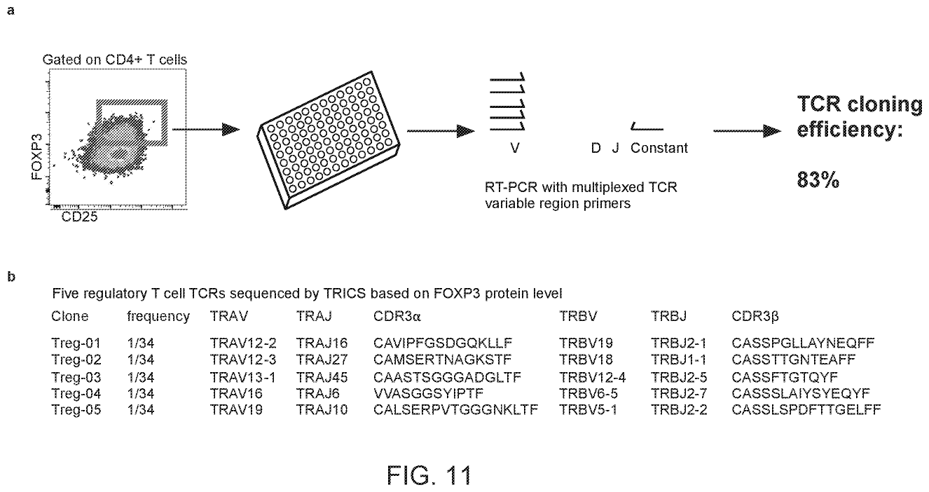

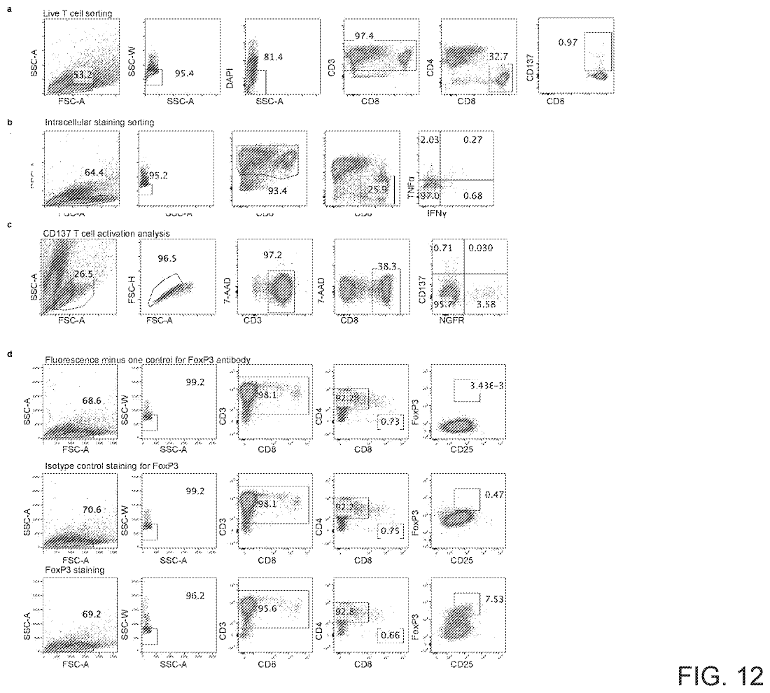

T cell receptor selection based on intracellular staining would be useful beyond just the capture of cytokine producing TCRs. Knowledge of Treg epitopes and reactivities has been lacking due to the need to phenotype Tregs exclusively by nuclear transcription factor FOXP3 (21). Indirect analysis had been performed by coupling multiple TCR analysis techniques, which showed capacity to recognize tumor antigens. To explore the ability of ICS based TCR sequencing in human Treg cells, we performed single-cell deposition of T cells that expressed the classic Treg markers: CD3, CD4, CD25 and FOXP3 (FIG. 11). TCR sequencing and analysis, following intracellular staining for FOXP3, showed remarkable efficiency of TCR cloning: 33 out of 40 single cells deposited returned productive alpha/beta pairs (83%) (FIG. 11). A specific peptide was not queried, thus the TCRs identified did not show any clonality, unlike the viral antigen specific CD8 T cells analyzed previously (FIG. 11). Profiling of both CD4⁺ Treg as well as CD8⁺ effector cell TCRs, showed that ICS based selection can identify TCRs across T cell phenotypes and functionalities.

### Single-Cell Sequencing of Fixed and Permeabilized Cells in Droplet-Based Format

In 2017 Macosko et al. published a seminal report, demonstrating that single-cell mRNA sequencing can be performed with remarkable efficiency in a fluid droplet (22). In our hands, PFA fixed cells performed poorly with a droplet-based sequencing approach. For singly deposited cells, we used heat to break the crosslinking between mRNA and protein to allow the RT reaction to proceed. Because this would not be possible in a droplet format, we had to develop a non-temperature-based method of crosslinking reversion. In view of this, we developed an ICS approach with a chemically reversible crosslinker, DSP (Lomant's Reagent, 3,3′-Dithiodipropionic acid di(Nhydroxysuccinimide ester)) (23). DSP reacts with primary amines, has a sulfide bond in the center, and can be cleaved via a reducing agent. Once a cell has been encapsulated into a droplet, mRNA can be released for cDNA generation. DSP has previously been used to preserve cells prior to single cell mRNA sequencing using the Fluidigm C1 machine (24). However, DSP has not been used to fix cells for ICS staining of cells in suspension.

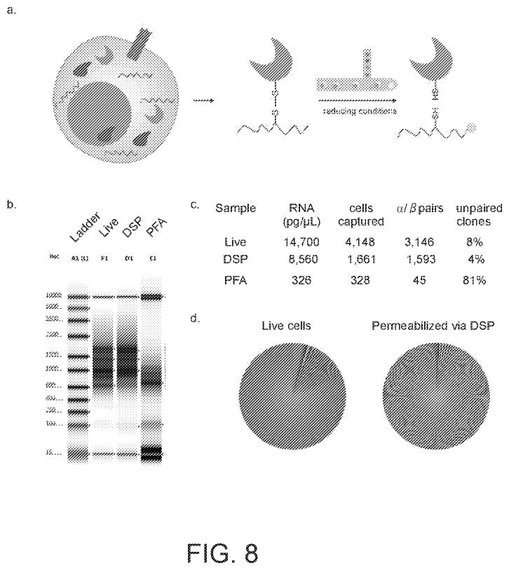

CLint-Seq was then used to sequence TCRs in bulk T cell population via the 10× genomics V(D)J library construction (FIG. 8a). The cDNA profile showed that DSP crosslinked cells are comparable to live cells, but PFA processed cells give a poor cDNA profile (FIG. 8b). Subsequently, these samples were sequenced and single-cell data analyzed (FIG. 8c). DSP crosslinked cells showed cDNA density comparable to live cell control of 8560 pg/ul and 14700 pg/ul respectively. However, PFA crosslinked cells had cDNA density of 326 pg/ul. Live cells also had a higher cell capture rate compared with DSP crosslinking at 4,148 cells and 1,661 cells respectively. Yet, PFA crosslinking allowed for just 328 cells to be identified. Further, the proportion of clones that contain both and alpha and a beta chain is indicative of the sample quality. DSP crosslinked cells contained only 4% of unpaired clones, compared to 8% of live cells. The PFA crosslinking yielded 81% of unpaired clones. Additionally, the diversity of live and DSP fixed and permeabilized T cells was similar (FIG. 8d). Thus, the cDNA generation, cell capture, frequency of unpaired clones and TCR diversity indicated single-cell gene expression can be performed in cells permeabilized via DSP crosslinking in a manner that is superior to conventional methodologies.

### CLint-Seq Coupled to Droplet-Based Sequencing Recovers EBV Specific TCRs

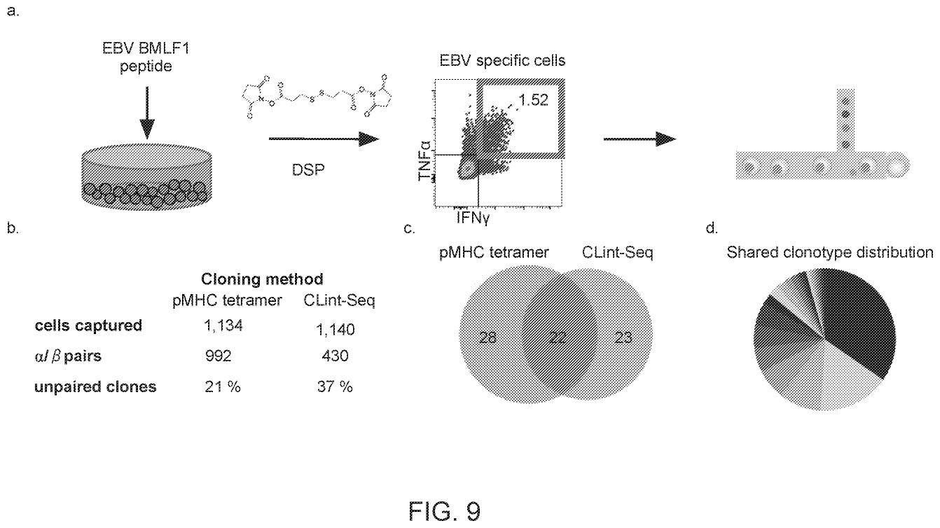

Properly controlled untethering of mRNA in the fluidics system can be difficult to achieve. If mRNA is released prior to cell encapsulation into a fluid droplet, then mRNA cellular origin will not be identified correctly. We subjected a population of EBV specific T cells selected by IFNγ and TNFα expression for 10× V(D)J sequencing and compared the clonotypes to live, tetramer selected cells. Both CLint-seq processed cells and live, tetramer selected cells returned a similar number of cells captured (FIG. 9b). Live cells showed greater recovery of alpha/beta pairs, as well as a lower frequency of unpaired clones 21% compared to 37%, respectively (FIG. 9b). To determine if mRNA identity is indeed maintained we compared clonotypes recovered by CLint-Seq to tetramer selected live cells. Two clonotype populations overlapped significantly, indicating faithful TCR clone recovery and general faithful mRNA recovery from single cells (FIG. 9c). The shared TCRs also represented a diversity of clonotype frequencies (FIG. 9d). This indicated that mRNA cellular origin is maintained when cells are processed per the CLint-Seq protocol.

### Methods:

### T Cell Cultures:

Peripheral blood mononuclear cells (PBMCs) from normal donors were used for most experiments, as previously described (11). For the analysis of the Cytomegalovirus (CMV) response we used leukapheresis product from a melanoma patient. TCRPMI is used in these experiments and contains 1640 RPMI (Thermo Fisher, cat. no. 31800089) supplemented with 10% Fetal Bovine Serum (Omega Scientific, cat. no. FB-11), 1× Glutamax (Thermo Fisher, cat. no. 35050061), IX sodium pyruvate (Thermo Fisher, cat. no. 11360070), 10 mM HEPEPS (Thermo Fisher cat. no. 15630130), 1× non-essential amino acids (Thermo Fisher, cat. no. 11140050) and 50 μM β-mercaptoethanol (Sigma-Aldrich, cat. no. M3148). Briefly cells were thawed into warm media and rested overnight at 10×10⁶ million/ml in 24 well plate (Corning cat. no. 353047) in TCRPMI media. Subsequently, cells are washed once and cultured for 9 days in TCRPMI supplemented with 1 ug/ml of antigenic peptide: CMV pp65 (NLVPMVATV), EBV BMLF1 (GLCTLVAML) (Elim Biopharmaceuticals Inc) and 25 Units/ml IL2 (Peprotech, cat. no. 200-02). Half of media is replaced every 2-3 days. For Regulatory T cell (TReg cell) analysis normal human PBMCs were used. CD4+ T cells were isolated using MACS beads (Miltenyi Biotec cat. no. 30-045-101), resuspended in TexMACS media (Miltenyi Biotec cat. no. 130-097-196) with 5% human AB serum and 500 Units/ml of human IL2 (Peprotech) at 1×106 cells/ml and aliquot 100 ul per well into a 96 well plate. Then MACSiBeads coated with CD3 and CD28 antibodies were added at a 4:1 ratio in 20 ul of TexMACS media with 5% human AB serum. Next day 100 ul of TexMACS with 5% human AB (Omega Scientific, cat. no. HS-20) and 500 Units/ml of IL2 were added to each well. Cells were expanded under these conditions by adding fresh media every three days. At day 9 Treg cells were washed, magnetically isolated from MACSiBeads and plated for 12 hour rest.

### Retroviral TCR Transduction:

Virus was produced as described previously (11). Normal donor PBMCs were thawed and stimulated with Dynabeads (Thermo Fisher, cat. no. 11132D) at 1:1 ratio in AIM V (Thermo Fisher, cat. no. 12055083) media with Human AB serum. 50 Units/ml of IL2, Glutamax and 50 μM β-mercaptoethanol. Stimulation is done in 24 well TC plate at 2×10⁶ cells/well. After 2 days about 1.5 ml of media was removed from each well and 1 ml of retroviral supernatant added with 5 ug/ml polybrene (Sigma-Aldrich, cat. no. H9268). Cells are then centrifuged at 1350 G for 90 minutes at 30 C. Post transduction about 1 ml was removed from each well and 1 ml of T cell media was added with 50 Units/ml of IL2. Next day viral transduction via centrifugation was repeated. The following day cells were washed once with T cell media and each well resuspended in 2 ml of T cell media. Our TCR constructs contain the murine constant region. Viral transduction was evaluated by truncated NGFR secondary marker staining with NGFR-PE (Biolegend cat. no. 345110).

### Intracellular Staining:

PBMCs are washed 2 times with PBS (Fisher Scientific cat. no. MT-46013CM) and once with TCRPMI. Then cells are resuspended in TCRPMI at 500,000 cells/100 μL of media and aliquoted into 96 well plate (Corning cat. no. 353077) for 12-hour rest prior to intracellular staining stimulation. Then, 100 μL of TCRPMI with 20 μg/ml of antigenic peptide and 2 μg/ml of CD28/49d antibodies (BD cat. no. 347690) are added to each well. AIM V complete media is used for TCR overexpression experiment. Cells are incubated for one hour at 37 C 5% CO₂ and 20 μL of 10× brefeldin A (Biologened cat no. 420601) is added to each well. Cells are further incubated for 8 hours. For TCR sequencing cells are stained immediately under RNAse free conditions with the FRISCR protocol adapted from Thomsen et al. Fixed single-cell transcriptomic characterization of human radial glial diversity. Nat. Methods 13, 87 (2016). Briefly each well is washed twice with 200 μL of wash buffer, which contains nuclease free water (Thermo Fisher, cat. no. 4387936), 10× molecular biology grade PBS, 1% nuclease free BSA (Gemini cat. no. 700-106P)), and 1:400 RNAsin Plus (Promega cat. no. N2615). For TCR overexpression and subsequent sequencing experiment we used RNAsin plus at 1:40000 dilution. The cells are stained with following surface antibodies: CD3-APCCy7 (Thermo Fisher, cat. no. 47-0036-42), CD8a-PE (Thermo Fisher, cat. no. 12-0088-42), CD4-PECy7 (Biolegend, cat. no. 300512). After staining at 4 C cells are washed with wash buffer and fixed with 100 μL of 4% PFA (EMS cat. no. 15710) for 10 minutes at 4 C. Then cells are washed twice and resuspended in 1% BSA buffer with 0.1% Triton X-100 (Sigma-Aldrich cat. no. T8787) for 10 minutes. Cells are then washed and subsequently stained with intracellular antibodies in wash buffer for IFNγ-APC (Biolegend cat. no. 506510). TNFα-FITC (Biolegend cat. no. 502906). FoxP3-A488 (Biolegend cat. no. 320012), msIgG1-A488 (Thermo Fisher, cat. no. MG120). Cells are then washed and resuspended in wash buffer for FACS analysis. Intracellular staining where we did not plan to do TCR sequencing was done in the absence of RNAsin plus inhibitor.

### Intracellular Staining for CLint-Seq

All buffers except crosslinker step contain 1:400 RNAsin (Promega) and molecular biology grade PBS to inhibit RNA degradation. Cells were first washed twice in 1% BSA (Gemini) buffer with 1:400 RNAsin (wash buffer) and incubated for 15 minutes on ice with surface antibodies: CD3-APCCy7 (Thermo Fisher, cat. no. 47-003642), CD8a-PE (Thermo Fisher, cat. no. 12-0088-42), CD4-PECy7 (Biolegend, cat. no. 300512). DSP is stored at −20 C in a desiccant filled container. Immediately prior to experiment DSP is left at room temperature for at least 30 minutes and then prepared to a concentration of 50 mg/ml in molecular biology grade DMSO (Sigma). Then 1 mg/ml solution is prepared in molecular biology grade PBS, by vortexing 20 ul of DSP in a 15 ml conical tube and adding 1 mL of PBS with P1000. DSP is filtered using a 40 μm Flowmi strainer (Sigma). Then 0.25 mg/ml solution is prepared in PBS. Cells are washed once in wash buffer and twice with PBS and resuspended in 200 μL of 0.25 mg/ml DSP (Thermo Fisher). Cells are incubated at room temperature for 30 minutes and quenched with 200 mM Tris (Thermo Fisher). Cells are then washed twice and incubated for 10 minutes with 100 μL of 0.05% Triton X-100 (Thermo Fisher) in wash buffer. Subsequently, cells are washed and resuspended in wash buffer for 20 minutes with antibodies: IFNγ-APC (Biolegend cat. no. 506510), TNFα-FITC (Biolegend cat. no. 502906). Then, cells are washed again and resuspended for FACS sorting.

### 10× Library Preparation:

10× genomics human V(D)J libraries were prepared by the UCLA Technology Center for Genomics & Bioinformatics per the typical 10× genomic library construction protocol without any modifications to this protocol.

### 10× Library Sequencing:

Single cell TCR libraries were sequenced by Illumina NextSeq. Data was analyzed using 10× genomics pipeline to generate Vloupe files.

### CD137 and Tetramer Staining:

PBMCs were either cultured with TCRPMI as described above and reported previously (11). For TCR overexpression experiments we used AIM V media as described previously. PBMCs were washed with PBS two times and once with media, subsequently resuspended at 5×10⁵ cells/100 μL and aliquoted in 96 well plate for 12 hour rest. Then, cells were stimulated with 20 μg/ml of antigenic peptide and 2 μg/ml of CD28/49d in 100 μL of media for 24 hours. PBMCs were then washed with wash buffer as described above, but RNAsin plus inhibitor was excluded. PBMCs were then stained with CD3-APCCy7 (Thermo Fisher, cat. no. 47-0036-42). CD8a-PE (Thermo Fisher, cat. no. 12-0088-42), CD4-PECy7 and CD137-APC (Biolegend cat. no. 309810) antibody for 20 minutes. Subsequently, cells were washed, resuspended in wash buffer and 7-AAD (BD cat. no. 559925) or DAPI was added immediately prior to FACS analysis or sorting. Tetramer staining was performed as previously described and MART-1 (ELAGIGILTV) HLA-A2 tetramer was made in-house (11). Tetramers for NY-ESO-1 (MBL cat. no. TB-M011-1), CMV pp65 (MBL cat. no. TB-0010-2). EBV BMLF1 (MBL cat. no. TB-M011-2) were purchased.

### FACS Single-Cell Deposition

Briefly, cells were gated on live lymphocytes by light scatter, single events, CD3⁺, CD8⁺, TNFα⁺/IFNγ⁺ or tetramer/CD137⁺ (FIG. 11). Background signal was either set on DMSO stimulation or irrelevant tetramer, to maximize detection of true positive events. Cells were then singly deposited into 96 well plates for TCR cloning or bulk sorted into DNA LoBind 2 ml tubes containing 400 μL of 0.04% BSA buffer with 1:400 RNAsin. Sorted cells are then resuspended at more than 150 cells/μL in 0.04% BSA with 1:400 RNAsin to be submitted for 10× library preparation.

### RT-PCR Analysis, and TCR Cloning:

A detailed protocol that can be adapted for our TCR cloning strategy is described (11). Antigen specific T cells were deposited at 1 cell/well into 96 well plate containing lysis buffer with One Step RT-PCR reagents (Qiagen cat. no. 210212). Plates were immediately placed on dry ice and then frozen at −80 C for further analysis. Subsequently, plates were thawed on ice and incubated at 56 C for 1 hour. This allowed for reverse cross linking of mRNA from protein. Each well was then split into two for independent sequencing of alpha and beta TCR chains. RT-PCR reaction was performed with multiplex TCR variable region primers (IDT) and alpha and beta constant region primers using Qiagen one step RT-PCR to generate TCR cDNA. Nested alpha and beta chain PCR was performed to amplify the TCR cDNA and the product was then sanger sequenced (Laragen Inc). Assembly PCR and restriction enzyme cloning was performed to generate the retroviral constructs, per the following map: tNGFR-P2A-TCRα-F2A-TCRβ.

### TCR Coculture for ELISA and Killing Assays:

We followed a previously published protocol for TCR functional validation via a cytotoxicity assay. Briefly, TCR transduced PBMCs were cocultured with target cell line PC3 that expressed HLA-A2 and pp65 CMV protein. Cocultures were set up at 2:1 E:T ratio in 100 ul of F12K media (ATCC cat. no. 30-2004) supplemented with 10% Fetal Bovine Serum and L-glutamine (Fisher Scientific cat. no. BP379-100) in 96 well, flat bottom plate. Cell killing was visualized using the IncuCyte system (Sartorius), which quantified GFP levels in PC3 cells. At 48 hours 50 ul of supernatant was collected and IFNγ ELISA was performed.

As illustrated above, CLint-Seq can be used for the discovery of TCRs that recognize tumor, viral, and self-antigens. By combining modular stimulation protocols with the specificity provided by cytokine production, TCR discovery with CLint-Seq can complement or replace current methods. Tetramer construction is laborious and relies on determining the peptide epitope, which is often done using prediction algorithms. Yet, prediction algorithms are known to predict false positive epitopes as well as miss real ones. Once made, MHC class I tetramers can work well for CD8⁺ T cells, however, it is difficult to construct MHC class II tetramers for CD4⁺ T cells. Once made they require very high affinity to detect CD4⁺ T cells (8). Therefore, tetramer-based detection of reactive CD4⁺ T cells is difficult. However, CD4⁺ T cells represent an important component of the cytotoxic T cell response and have been shown to direct antitumor responses in multiple models (10). Epitope mapping field has used libraries of overlapping peptides to unbiasedly determine epitopes to which there is a T cell response. This approach often used ICS as a read out. Thus, CLint-Seq can be coupled with T cells stimulation by a peptide library to sequence the TCRs of the responding population. Rapid TCR identification is necessary in personalized T cell immunotherapy, as the cellular product needs to be prepared before the patient succumbs to the disease. Currently, such pipelines rely on tetramer or activation marker techniques. It's possible to generate peptide pool that cover an antigenic region and stimulate autologous PBMCs or tumor infiltrating lymphocytes. Such strategy would capture tumor reactive CD4⁺ T cells as well as full breadth of CD8⁺ T cells. This will result in a greater number of antigen reactive TCRs per patient, as well as identify a higher number of patients that are suitable for personalized therapy engineering.

Regulatory T cell adoptive cell therapy is being advanced to the clinic by multiple groups. The goal is to limit or suppress effector responses to self-antigens, which mediate autoimmune disease. Because not many Treg TCRs have been described, these therapies are not antigen specific. We have shown that CLint-Seq can be used for identification of Treg TCRs based on FOXP3 intracellular staining. This selection can be enhanced by either including additional transcription factors or intracellular cytokines. For example, the Helios transcription factor can help identify Treg cells that differentiate in the thymus rather than the peripheral tissue and thus are truly self-antigen reactive. For antigen-specific selection of self-reactive Treg TCRs its possible to perform peptide based stimulation and subsequent cytokine based selection of reactive TCRs. Thus rather than using polyclonal Tregs that may cause broad immune suppression, CLint-Seq can help identify TCRs that can help adoptive cell therapy home to a specific pathological site in the body to treat a specific autoimmune condition.

CLint-Seq allows for droplet based single-cell mRNA sequencing. Any cell type can be stained for multiple intracellular antigens and cells can be sorted by FACS for the desired phenotype and subsequently single cell sequenced. Global mRNA sequencing at the single-cell allowed for definition new phenotypes and drug targets. However, proteins are the functional units of cells. Therefore, mRNA sequencing has major limitations. For example, some important proteins with long half-lives will have very low mRNA abundance (26). Thus, it is difficult to use mRNA sequencing to detect such proteins and consequently define cell phenotypes. Alternatively, mRNA and protein can be globally quantified by staining cells with antibody-oligo complexes. Techniques such as CITE-seq and REAP-seq allow for simultaneous detection of protein and mRNA in single cells using oligo tagged antibodies and single cell sequencing (27, 28). However, this analysis is limited to surface proteins as the assumption is that mRNA would become degraded upon ICS with Antibody-oligo constructs. Our method of cell crosslinking using DSP and permeabilization will permit simultaneous proteomics and transcriptomics on a single-cell level.

In summary, we define a new method for generating antigen specific TCRs and confirm its validity with CMV and EBV CD8 T cell responses. This technology is immediately applicable to high throughput TCR characterization and can lead to the development of novel adoptive cell therapies. However, the particular advance of sequencing mRNA in fixed and permeabilized cells will be applicable across cell biology to understand single cell level behavior at both protein and gene expression levels.

### Example 3 References

1. N. P. Restifo. M. E. Dudley, S. A. Rosenberg, Adoptive immunotherapy for cancer: Harnessing the T cell response. Nat. Rev. Immunol. 12, 269-281 (2012).
2. T. N. M. Schumacher, T-cell-receptor gene therapy. Nat. Rev. Immunol. 2, 512-519 (2002).
3. C. Raffin. L. T. Vo, J. A. Bluestone, Treg cell-based therapies: challenges and perspectives. Nat. Rev. Immunol. 20, 158-172 (2020).
4. P. F. Robbins, et al., Tumor regression in patients with metastatic synovial cell sarcoma and melanoma using genetically engineered lymphocytes reactive with NY-ESO-1. J. Clin. Oncol. 29, 917-924 (2011).
5. T. Feuchtinger, et al., Adoptive transfer of pp65-specific T cells for the treatment of chemorefractory cytomegalovirus disease or reactivation after haploidentical and matched unrelated stem cell transplantation. Blood 116, 4360-4367 (2010).
6. C. Rius, et al., Peptide-MHC Class I Tetramers Can Fail To Detect Relevant Functional T Cell Clonotypes and Underestimate Antigen-Reactive T Cell Populations. J. Immunol. 200, 2263-2279 (2018).
7. L. V Sibener, et al, Isolation of a structural mechanism for uncoupling T cell receptor signaling from peptide-MHC binding. Cell 174, 672-687. e27 (2018).
8. J. J. Sabatino, J. Huang, C. Zhu, B. D. Evavold, High prevalence of low affinity peptide-MHC II tetramer-negative effectors during polyclonal CD4+ T cell responses. J. Exp. Med. 208, 81-90 (2011).
9. E. Stronen, et al., Targeting of cancer neoantigens with donor-derived T cell receptor repertoires. Science (80-.). 352, 1337-1341 (2016).
10. C. Linnemann, et al., High-throughput epitope discovery reveals frequent recognition of neo-antigens by CD4+ T cells in human melanoma. Nat. Med. 21, 81 (2014).
11. M. Wolfl, et al., Activation-induced expression of CD137 permits detection, isolation, and expansion of the full repertoire of CD8+ T cells responding to antigen without requiring knowledge of epitope specificities. Blood 110, 201-210(2007).
12. M. R. Betts, et al., Sensitive and viable identification of antigen-specific CD8+ T cells by a flow cytometric assay for degranulation. J. Immunol. Methods 281, 65-78 (2003).
13. H. Brosterhus, et al., Enrichment and detection of live antigen-specific CD4+ and CD8+ T cells based on cytokine secretion. Eur. J. Immunol. 29, 4053-4059 (1999).
14. Q. Ye, et al., CD137 accurately identifies and enriches for naturally occurring tumor-reactive T cells in tumor. Clin. Cancer Res. 20, 44-55 (2014).
15. S. L. Waldrop, C. J. Pitcher, D. M. Peterson, V. C. Maino, L. J. Picker,
Determination of antigen-specific memory/effector CD4+ T cell frequencies by flow cytometry: evidence for a novel, antigen-specific homeostatic mechanism in HIV-associated immunodeficiency. J. Clin. Invest. 99, 1739-1750 (1997).
16. C. J. Pitcher, et al., HIV-1-specific CD4+ T cells are detectable in most individuals with active HIV-1 infection, but decline with prolonged viral suppression. Nat. Med. 5, 518 (1999).
17. M. K. Slifka, F. Rodriguez, J. L. Whitton, Rapid on/off cycling of cytokine production by virus-specific CD8+ T cells. Nature 401, 76 (1999).
18. M. T. Bethune, et al., Isolation and characterization of NY-ESO-1-specific T cell receptors restricted on various MHC molecules. Proc. Natl. Acad. Sci. 115, E10702-E10711 (2018).
19. E. R Thomsen, et al., Fixed single-cell transcriptomic characterization of human radial glial diversity. Nat. Methods 13, 87 (2016).
20. P. Klenerman, A. Oxenius, T cell responses to cytomegalovirus. Nat. Rev. Immunol. 16, 367 (2016).
21. M. Ahmadzadeh, et al., Tumor-infiltrating human CD4+ regulatory T cells display a distinct TCR repertoire and exhibit tumor and neoantigen reactivity. Sci. Immunol. 4, eaao4310 (2019).
22. E. Z. Macosko, et al., Highly parallel genome-wide expression profiling of individual cells using nanoliter droplets. Cell 161, 1202-1214 (2015).
23. K. Subramonia Iyer, W. A. Klee, M. Wooi, “Direct Spectrophotometric Measurement of the Rate of Reduction of Disulfide Bonds ‘THE REACTIVITY OF THE DISULFIDE BONDS OF BOVINE cr-LACTALBUMIN” (1973).
24. M. Attar, et al., A practical solution for preserving single cells for RNA sequencing. Sci. Rep. 8, 1-10 (2018).
25. J. F. Ashouri, A. Weiss, B Cells Antigen Receptor Signaling in Human T and Endogenous Nur77 Is a Specific Indicator of (2016) https:/doi.org/10.4049/jimmunol.1601301 (May 9, 2020).
26. B. Schwanhausser, et al., Global quantification of mammalian gene expression control (2013) https:/doi.org/10.1038/nature10098.
27. M. Stoeckius, et al., Simultaneous epitope and transcriptome measurement in single cells. Nat. Methods 14, 865-868 (2017).
28. V. M. Peterson, et al., Multiplexed quantification of proteins and transcripts in single cells. Nat. Biotechnol. 35, 936-939 (2017).

All publications mentioned herein (e.g. those references numerically listed above) are incorporated herein by reference to disclose and describe aspects, methods and/or materials in connection with the cited publications.

1. A method of crosslinking and permeabilizing mammalian cells, the method comprising combining the mammalian cells with:
a permeabilization agent; and
a chemically cleavable crosslinker selected to:
couple intracellular polypeptides to mRNA; and
release the cellular polypeptides from mRNA under reducing conditions;
such that the mammalian cells are permeabilized and crosslinked.

2. The method of claim 1, further comprising:
combining the fixed and permeabilized mammalian cells with fluorescent antibodies directed to one or more target polypeptides within the fixed and permeabilized mammalian cells;
performing fluorescent activated cell sorting to select one or more fixed and permeabilized mammalian cells containing the one or more target polypeptides;
sequencing one or more mRNAs present in the one or more selected mammalian cells.

3. The method of claim 2, wherein the method comprises:
releasing the cellular polypeptides from mRNA under reducing conditions; and/or
encapsulation of the mammalian cells within fluid droplets; and/or
combining the mammalian cells with beads comprising barcodes; and/or
obtaining the sequences of one or more mRNAs present in the one or more selected dead mammalian cells using a dynamic microfluidic system.

4. The method of claim 1, wherein the T cells are combined with a chemically cleavable crosslinker selected to:
couple mRNA to intracellular polypeptides within FOXP3 expressing cells; and
release the intracellular polypeptides from mRNA under reducing conditions.

5. The method of claim 1, wherein the chemically cleavable crosslinker comprises 3,3′-Dithiodipropionic acid di(Nhydroxysuccinimide ester).

6. The method of claim 5, wherein the method uses 3,3′-Dithiodipropionic acid di(Nhydroxysuccinimide ester) in amounts from about 0.01 mg/ml to about 10.0 mg/ml.

7. The method of claim 1, wherein the fluorescent antibodies are selected to bind one or more cytokines observed to be produced in activated T cells; and/or FOXP3.

8. The method of claim 1, wherein the permeabilization agent comprises triton-X100.

9. The method of claim 3, wherein releasing the cellular polypeptides from mRNA under reducing conditions comprises adding a reducing agent the cells.

10. A composition of matter comprising mammalian cells produced by a method of claim 1.

11. A method for obtaining polynucleotides encoding Vα and Vβ T cell receptor polypeptides comprising:
combining together an antigen presenting cell, antigen, T cells and a cytokine secretion inhibitor under conditions selected to activate the T cells in response to the antigen;
fixing and permeabilizing activated T cells;
combining the fixed and permeabilized T cells with fluorescent antibodies directed to one or more cytokines observed to be produced in activated T cells that are present within the fixed and permeabilized T cells;
performing fluorescent activated cell sorting to select one or more cells containing the one or more cytokines observed to be produced in activated T cells;
obtaining polynucleotides encoding Vα and Vβ T cell receptor polypeptides from the selected one or more cells.

12. The method of claim 11, wherein a single fixed and permeabilized dead T cell is selected by fluorescent activated cell sorting.

13. The method of claim 12, wherein polynucleotides encoding Vα and Vβ T cell receptor polypeptides are obtained from the single cell using a polymerase chain reaction process.

14. The method of claim 11 wherein the antigen:
is associated with a human leukocyte antigen on an antigen presenting cell;
comprises a plurality of different antigens (e.g. a plurality of different antigenic peptides); and/or
comprises a peptide-MHC tetramer.

15. The method of claim 11, wherein the cytokine secretion inhibitor comprises Brefeldin A.

16. The method of claim 11, wherein the one or more cytokines include TNFα and/or IFNγ.

17. The method of claim 11, wherein the T cells are obtained from primary peripheral blood mononuclear cells.

18. The method of claim 11, wherein the T cells are obtained from an individual diagnosed with a pathological condition.

19. The method of claim 18, wherein the pathological condition is cancer.

20. A composition of matter comprising polynucleotides encoding Vα and Vβ T cell receptor polypeptides obtained by a method of claim 11.


SEQUENCE LISTING (3 sequences)

SEQ ID NO: 1 - protein, 10 aa, Artificial Sequence
  Description of Artificial Sequence Synthetic peptide
ELAGIGILTV

SEQ ID NO: 2 - protein, 9 aa, Artificial Sequence
  Description of Artificial Sequence Synthetic peptide
NLVPMVATV

SEQ ID NO: 3 - protein, 9 aa, Artificial Sequence
  Description of Artificial Sequence Synthetic peptide
GLCTLVAML